(19) United States
(12) Patent Application Publication
(10) Pub. No.: US 2024/0269101 A1
(43) Pub. Date: Aug. 15, 2024

(54) COMPOSITIONS COMPRISING MEDIUM ODD CHAIN LIPIDS WITH MULTIFUNCTIONAL ACTIVITY TO PREVENT AND TREAT SKIN INFLAMMATION AND AGING

(71) Applicant: EXCELLA BIOSCIENTIFIC LTD, Nahariya (IL)

(72) Inventor: Sabina GLOZMAN, Nahariya (IL)

(73) Assignee: EXCELLA BIOSCIENTIFIC LTD, Nahariya (IL)

(21) Appl. No.: 18/417,470

(22) Filed: Jan. 19, 2024

Related U.S. Application Data
(63) Continuation-in-part of application No. PCT/IL2022/050777, filed on Jul. 19, 2022.
(60) Provisional application No. 63/265,310, filed on Dec. 13, 2021.
(60) Provisional application No. 63/223,322, filed on Jul. 19, 2021.

(51) Int. Cl. A61K 31/23; A61K 8/37; A61K 9/00; A61K 45/06; A61P 17/02; A61P 17/10; A61Q 19/08

(52) U.S. Cl. CPC A61K 31/23; A61K 8/37; A61K 9/0014; A61K 45/06; A61P 17/02; A61P 17/10; A61Q 19/08

(57) ABSTRACT

New products derived from triazelaic glycerols, products derived from naturally occurring sources of unsaturated fatty acids, compositions, manufacturing processes, and uses in various disease conditions associated with Microbiome alterations and other inflammatory processes are provided.

### FIELD OF THE INVENTION

The present invention relates to products derived from naturally occurring sources of unsaturated fatty acids, compositions, manufacturing processes, and uses in various skin conditions associated with inflammation, aging, and microbiome alterations.

### BACKGROUND OF THE INVENTION

Being exposed to continuous external and internal assaults, skin requires constant maintenance and care. Skin barrier changes are accountable for a wide variety of conditions with inflammatory disorders, with acne being the most abundant, all the way through the anti-aging. In general, any abnormality of the skin can be attributed to multiple interconnected factors and thus require complex and creative treatment means. While healing a certain skin disorder, inaccurate or aggressive treatment may trigger various pathologies and cause significant damage.

Lipases, are virulent factors that released from opportunistic microbiome of the skin, e.g. Cutibacterium acnes and Staphylococcus species, Candida spp, Malazzeria spp and invading pathogens, such as resistant nosocomial strains, e.g. Staphylococcus MRSA, Pseudomonas spp., fungi-Aspergillus spp. Under unbalanced microbiome or under invasion of pathogenic species, overexpressed virulent lipases hydrolyse lipids in epidermal cell membrane of skin barrier into peroxidation prone polyunsaturated free fatty acids-FFA that lead to formation of Lipid Peroxides (LPO) and cyclooxygenase and lipoxygenase-mediated formation of leukotrienes and prostaglandins-so initiating oxidative stress and inflammatory molecular signaling cascade.

Azelaic acid is an approved drug and cosmetic ingredient known to be used as anti-inflammatory, anti-acne, anti-melasma, anti-rosacea, skin lightening active ingredient in topical cosmetic and medical topical formulations. Similarly, to the retinoic and lipo-hydroxy acids, azelaic acid have side effects, such as irritation. Antibiotics are also broadly used but not selective to lipase overexpressing species therefore damage healthy microbiome of the skin leading to unbalanced microbiome (less diversity) and dysbiosis. However, when applied topically, the antibiotics can lose their effectiveness if used for a prolonged period due to resistance developed by the bacteria. The oral administration of antibiotics subjects the entire to body the antibiotic composition; instead of targeting the affected area. almost all antibiotics have undesirable side effects when taken orally and systemic dysbiosis. Thus, effective and safe treatment means for disorders mediated by lipase producing pathogens remain a long and unmet need.

### SUMMARY OF THE INVENTION

It is an object of the present invention to provide effective and safe therapeutics and delivery systems for health conditions associated with activity of lipases secreted by microorganisms.

It is another object of the present invention to provide effective and safe therapeutics for various skin abnormalities associated with inflammation, microbiome disbalance and/or aging.

According to some embodiments, the invention provides topical anaplerotic compositions based on C9 Medium chain fatty acids (C9 MOCFA) and derivatives thereof for the treatment of skin abnormalities associated with inflammation, microbiome disbalance and/or aging.

According to some embodiments, the invention provides topical anaplerotic compositions based on C9 MOCFA and derivatives thereof for the treatment of skin abnormalities associated with aterations in sebaceous microbiome.

According to some embodiments, the invention provides topical anaplerotic compositions based on C9 MOCFA and derivatives thereof for the treatment of skin abnormalities associated mitochondrial dysfunction.

According to some embodiments, the invention provides topical anaplerotic compositions based on C9 MOCFA and derivatives thereof for preventing skin abnormalities associated impaired lipogenesis and mitochondrial dysfunction.

According to some embodiments, the invention provides topical anaplerotic Triglecyride (TG) lipids-based and fatty acids (FA) precoursors enriched compositions for preventing and/or improving and/or trating and/or impaired repairing lipidome and mitichondrial dysfuntion.

According to some embodiments, the invention provides anaplerotic topical compositions comprising Triolein in carrier oil and a combination of Medium Odd Chain Tryglicerydes (MOCT) with MOCFA.

According to some embodiments, the invention provides a sebum replacement therapy based on active anaplerotuc compositions.

According to some embodiments, the invention provides compositions comprising TriAza for the treatment of skin inflammation, skin disorders associated with activity of lipases secreted by microorganisms, and impaired microbiome.

According to some embodiments, the invention provides novel methods of TriAza synthesis based on the desaturation and oxidation of Triolein, a major triglyceride of olive oil, for production of Triglyceride-like compounds.

According to some embodiments, upon the exposure of TriAza and its salts to bacteria-associated lipase, azelaic acid disassociates from the molecule and exhibits specific and selective to lipase targeting activity.

According to some embodiments, the invention provides methods for the preparation of such triazelaic-derived products and to uses of such triazelaic-derived products.

According to some embodiments, the invention provides effective and safe treatment means for skin disorders mediated by lipase producing pathogens.

According to some embodiments, the invention provides compositions comprising triazelaine for balancing human microbiome.

According to some embodiments, the invention provides compositions for treating disease or condition associated with disbalance of microbiome.

According to some embodiments, the invention provides topical compositions effective for the treatment of skin disorders, for balancing and maintaining skin microbiome, for slowing progression of undesired skin conditions such as, without limitation, aging, pigmentation, disorders of the philobaceous unit, and other conditions which may benefit from the compositions of the invention.

According to some embodiments, the invention provides a multifunctional carriers for topical compositions that can be used, without limitation, for skin conditioning functions, as a precursor for azelaic acid, and as a delivery vehicle for biomolecules and microbiome samples.

According to some embodiments, the invention provides a topical composition comprising triazelaine and, optionally, a carrier.

According to some embodiments, the invention provides a method of treating an inflammatory condition of the skin in a subject in need of such treatment, comprising topically administering to the subject an amount of triazelaine effective to treat said condition.

According to some embodiments, the invention provides a prodrug having the formula:

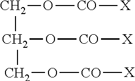

wherein X is azelaic acid, and wherein upon exposure to a bacterial lipase, azelaic acid is cleaved to become released from said compound.

According to some embodiments, the invention provides a topical composition for the treatment of a skin disorder comprising an active therapeutic agent and triazelaine as a carrier.

According to some embodiments, the invention provides a topical composition for skincare comprising triazelaine.

According to some embodiments, the invention provides a composition designed to deliver at least one biomolecule and/or chemical entity and/or biological sample to a subject in need of such delivery, comprising an amount of a carrier having Formula 1

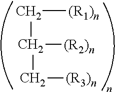

Wherein each of R₁, R₂, and R₃ is independently selected from the group consisting of dicarboxylic acid, azelaic acid; and wherein n is=/>1, and wherein the amount of said carrier is effective to deliver said biomolecule and/or chemical entity/and/or biological sample to a predetermined target.

According to some embodiments, the invention provides a compound of Formula 1

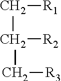

wherein each R₁, R₂, and R₃ is independently selected from the group consisting of azelaic acid or a derivative thereof, retinoic acid or a derivative thereof, succinic acid, lipophilic chain antioxidant, valproic acid, hydroxybutyric acid, nicotinamide, sebacic acid or a derivative thereof, linoleic acid, butyric acid, gamma-linoleic acid or a derivative thereof, alpha-hydroxy acids or a derivative thereof, cis-isomers of fatty acid, lactic acid, and zinc; and wherein upon exposure to a bacterial lipase, each of R₁, R₂, and R₃ is cleaved to become released from said compound.

According to some embodiments, the invention provides a compound of Formula 1

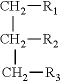

designed to deliver at least one chemical entity to a subject in need of the delivery of said at least one chemical entity, wherein each R₁, R₂, and R₃ is independently selected from the group consisting of active pharmaceutical or biological agent that released upon exposure to triglyceride lipase; and wherein upon exposure to a microbial lipase, each of R₁, R₂, and R₃ is cleaved to become released from said compound.

According to some embodiments, the invention provides a method of delivering at least one chemical entity and/or biomolecule into a food product for consumption or a raw material for the preparation of said food product, comprising adding to said food product or the raw material for the preparation of said food product an effective amount of the composition according to the embodiments of the invention.

According to some embodiments, the invention provides a method of treating a disorder in a subject in need of such treatment comprising administering to the subject an effective amount of the composition according to the embodiments of the invention.

### BRIEF DESCRIPTION OF THE DRAWINGS

FIG. 1 demonstrates a schematic pathway for the synthesis of azelaic acid glyceryl triester;

FIG. 2 demonstrates an alternate schematic pathway for synthesis of azelaic acid glyceryl triester;

FIG. 3 demonstrates a schematic pathway for synthesis of retinoic acid glyceryl triester;

FIG. 4 demonstrates a schematic pathway for the synthesis of 1,2-retinoic-3-azelaic acid glyceryl triester;

FIG. 5 demonstrates lipase-mediated dose-dependent activity measured by release of Azelaic acid from representative batch (L-PABS) in-vitro;

FIG. 6 demonstrates P. acnes-associated lipase-mediated activity of time-dependent release (day 1 and day 5 in the culture), of Azelaic acid form representative batches (L-PABS) under incubation with live culture of Corynobacterium acnes (P. acnes) in comparison to non-hydrolized L-PABS without exposure to bacteria enzyme in culture medium;

FIG. 7 demonstrates lipase-mediated sensitivity to hydrolysis by Triglyceride Lipase (Lipase) and release of Azelaic acid (A) comparison of batches (L-PABS) obtained by oxidative ozonolysis (Oz.) and by chemical synthesis (C.S.)—selectivity by hydrolysis by lipase-L and non-sensitivity to esterase-E treatment; (B) dose dependent sensitivity and selectivity of Azelaic acid release by Triglyceride Lipase, but not by Phospholipase, from demonstrating batches (L-PABS)

FIG. 8 demonstrates typical chromatogram of Olive oil;

FIG. 9 demonstrates typical chromatogram of Trioleate;

FIG. 10 demonstrates chromatogram of Pelargonic Acid in methanol by HPLC;

FIG. 11 demonstrates chromatogram of 0.5 mg/mL Pelargonic Acid standard obtained with GC method;

FIG. 12 demonstrates resulted products of the reaction. Left—H₂O₂ in excess to KMnO₄. Right—KMnO₄ in excess to H₂O₂;

FIG. 13 demonstrates olive oil before (top) and after (bottom) reaction with KMnO₄;

FIG. 14 demonstrates sample after 3 h of reaction;

FIG. 15 demonstrates sample after addition of large amount of water;

FIG. 16 demonstrates sample after reaction of KMnO₄ with Olive Oil in the presence of 1 g of selected surfactant-Brij-35;

FIGS. 17A-17D demonstrate: FIG. 17A—Before reaction; FIG. 17B—after reaction, extraction to Hexane. FIG. 17C—after reaction, extraction to Methanol. FIG. 17D—Preparation in Acetonitrile:Chloroform (90:10);

FIG. 18 demonstrates production process oxidative system variables tested in different batches;

FIG. 19 demonstrates different feeding acids and methods of obtained by oxidative ozonolysis used and compared with liquid synthesis;

FIG. 20 demonstrates qualification of triazelaine reaction end-products in NMR and MS spectrographs (peaks correlated to 602); and,

FIG. 21 demonstrates TLC results confirmed by MS in which TriAza end product and other by-products retention time on TLC was detected in various batches to select optimal parameters of production (batches: B1, OU3, B6, B13 and B14).

FIG. 22 demonstrates an alternate schematic pathway for synthesis of azelaic acid glyceryl triester using liquid esterification synthesis;

FIG. 23 demonstrates colorimetric assay on solid medium: upper picture—emulsified Triolein on the spot allows bacterial colonization). Bottom: negative control—no growth on agar only (left), no growth on emulsifier only (right);

FIG. 24 demonstrates colorimetric assay in liquid medium measuring non-purified enzyme secreted from C. acnes culture.

FIG. 25 demonstrates representative qualitative and quantitative analytical results characterize relative recovery of C9 Triaza derivatives of ozonolysis reaction including: OOO-ozonides, AzaA-azelaic acid, Pelargonic acid/PA, TriAza-TG9-derivatives.;

FIG. 26 demonstrates selection of TLC separation method by analysis of spot distribution in different running solvents post-ozonolysis (OOO-ozonides, TG9—TriAza, Pel—pelargonic acid, Az—azelaic acid, Olei—oleic acid, aa/ac acid—acetic acid used as a solvent.), and,

FIG. 27 demonstrates Screening antibacterial efficacy of C9-OCFA (azelaic-Az. Acid and Pelargonic-Pelarg. Acid) and MOCT-TriAza (batch VA4-1) with and without emulsifier

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to some embodiments, the invention provides a composition designed to deliver at least one biomolecule and/or chemical entity/and/or biological sample to a subject in need of such delivery, comprising an amount of a carrier having Formula 1

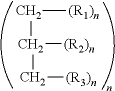

Wherein each of R₁, R₂, and R₃ is independently selected from the group consisting of dicarboxylic acid, azelaic acid; and wherein the amount of said carrier is effective to deliver said biomolecule and/or chemical entity/and/or biological sample to a predetermined target. In the context of the invention, the term “target” is meant to be understood as any tissue, organ, matrix, media, cell, organelle, or any other target that may benefit from the carrier. In the context of the invention, the term “biomolecule” refers, without limitation, to any molecular entity having biological activity as defined above. As used herein, the term “chemical entity” refers, without limitation, to a physical entity of interest in chemistry including molecular entities, parts thereof, and chemical substances. As used herein, the term “biological sample” refers, without limitation, to any material, including without limitation, blood, serum, fluid and tissue samples, collected from subjects and any tangible material directly or indirectly derived there from. In one embodiment, the carrier fulfills the function of a preservative.

According to some embodiments of the above composition, the composition may be, without limitation, an anti-septic, composition, a chemical composition, a pharmaceutical composition, a cosmeceutical composition, an edible composition, and a composition comprising microbiome. In one embodiment, the composition is a liquid composition. In one embodiment, the composition is a solid composition. In one embodiment, the composition is semi-solid composition.

According to some embodiments of the above composition, the unlimited list of compositions of the invention includes syrup, emulsion, powder, suspension, dispersion, cream, ointment, transdermal patch, a tablet, a capsule, a paste, a lotion, a soap, a surfactant-containing cleanser, oil, a powder foundation, an emulsion foundation, a wax foundation, a spray, and, a cosmetic base.

According to some embodiments, the invention provides a compound of Formula 1

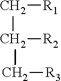

wherein each R₁, R₂, and R₃ is independently selected from the group consisting of azelaic acid or a derivative thereof, retinoic acid or a derivative thereof, succinic acid, lipophilic chain antioxidant, sebaceous acid or a derivative thereof, linoleic acid, gamma-linoleic acid or a derivative thereof, alpha-hydroxy acids or a derivative thereof, cis-isomers of fatty acid, lactic acid, vanillin, and zinc; and wherein, upon exposure to a bacterial lipase, each of R₁, R₂, and R₃ is cleaved to become released from said compound. As used herein, the term “lipophilic chain antioxidant” refers, without limitation, to a non-limiting list of lipophilic chain antioxidants of the invention include beta-carotene, vitamin E and lipoic acid, retinoic acid, terpene, phyto-protectants, medium or short chain fatty acid, alpha hydroxy acids, hydroxy-acids, polyol, sugar, oligosaccharides, polysaccharides, glycosides, salycilic acid, jasmonates, stilbenes niacinamides, biopeptides, proteins, chelators, bakuchiol, mannitol, zinc gluconate, hormone, nucleoside (olionucleotide), neurotransmitter, cannabinoid, etc.

According to some embodiments, the invention provides a compound of Formula 1

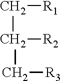

designed to deliver at least one chemical entity to a subject in need of the delivery of said at least one chemical entity, wherein each R₁, R₂, and R₃ is independently selected from active pharmaceutical, or biological, or metabolic agent, and wherein upon exposure to a microbial lipase, each of R₁, R₂, and R₃ is cleaved to become released from said compound. In the context of the invention, the non-limiting list o list of active pharmaceutical, or biological, or metabolic includes phytoprotectant, protective natural transmitter or metabolite, cosmetic agent, anti-ageing agent, microbiome regulating agent. As used herein, the term “microbial” refers without limitation to relating to or characteristic of a microorganism, especially a bacterium causing disease or fermentation. The unlimited list of microbials of the invention include bacteria, fungi, unicellular parasite.

According to some embodiments of the above compound, each of the of R₁, R₂, and R₃ independently has the formula

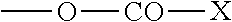

Wherein X is selected from the group consisting of azelaic acid or other active agent to be delivered through controlled release of microbiota associated lipase. In one embodiment, R₁, R₂, and R₃ are similar to each other. In one embodiment, the compound has the structure

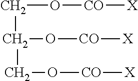

wherein X is azelaic acid. In one embodiment, the compound has the structure

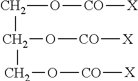

wherein X is retinoic acid. In one embodiment, the compound has the structure

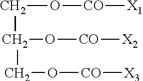

wherein X₁ and X₂ are both retinoic acid, and X₃ is azelaic acid. In one embodiment, the compound has the structure.

According to some embodiments, the invention provides a composition comprising the compound according to the one or more of the above embodiments, and at least one carrier.

According to some embodiments of the above composition, a non-limiting list of the compositions of the invention includes anti-septic composition, a chemical composition, a pharmaceutical composition, a cosmeceutical composition, an edible composition, and a composition comprising microbiome. In the context of the invention, the term “microbiome” refers, without limitation, to the microorganisms in a particular environment (including the body or a part of the body). Microbiome also refers to the combined genetic material of the microorganisms in a particular environment. Microbiome is considered as a term that describes the genome of all the microorganisms, symbiotic and pathogenic, living in and on all vertebrates. A non-limiting example of microbiome includes Lactobacillus, Bacillus, ammonia oxidizing bacteria, Coccus, and Bifidum, and balance of host microbiota or/and transplanted administered probiotic compositions that not overexpress lipase as a major virulence factor to induce biofilms and opportunistic and invading, contamination induced pathogens. According to some embodiments of the compositions of the invention, a non-limiting list of the compositions of the invention includes compositions suitable for oral administration, transdermal administration, topical administration, transmucosal administration, intranasal administration, ocular administration, mucosal administration, and vaginal administration.

According to some embodiments of the compositions of the invention, a non-limiting list of the compositions of the invention includes syrup, powder, suspension, emulsion, cream, ointment, transdermal patch, a suppository, drops, spray, foam, soap, shampoo, oil, mil, and colloid compositions.

According to some embodiments, the invention provides a method of treating a condition in a subject in need of such treatment comprising administering to the subject an effective amount of the composition according to the embodiments of the invention. In one embodiment, the condition is selected from skin diseases, overgrowth of lipase producing microorganisms, inflammation, melasma, melanoma, and hair loss. In one embodiment, said condition is a disorder of the pilosebaceous unit. In one embodiment, the condition is a malfunction of the immune system. In one embodiment, the condition is a disorder of the skin. In one embodiment, the condition is a disorder of the gastrointestinal tract.

According to some embodiments, the invention provides a topical composition comprising triazelaine and, optionally, a carrier.

According to some embodiments, the invention provides a method of treating an inflammatory condition of the skin in a subject in need of such treatment, comprising topically administering to the subject an amount of triazelaine effective to treat said condition.

According to some embodiments, the invention provides a prodrug having the formula:

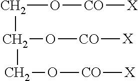

wherein X is azelaic acid, and wherein upon exposure to a bacterial lipase, azelaic acid is cleaved to become released from said compound.

According to some embodiments, the invention provides a topical composition for the treatment of a skin disorder comprising an active therapeutic agent and triazelaine as a carrier.

According to some embodiments, the invention provides a topical composition comprising triazelaine. Triazelaine is a glycerol of azelaic acids, and, in the context of the invention can also be called TriAza. In the context of the invention, the terms Triazelaine, TriAza of azelaic acids are and glycerol interchangeable.

According to some embodiments of the above composition, the composition comprises at least one carrier. In the context of the invention, a non-limiting list of carriers includes inactive ingredient, such as, without limitation, an excipient, pH buffering agent, filler, disintegrant, lubricant, thickener, surfactant, adjuvant, or any other cosmetic or pharmaceutical adjuncts conventionally employed in such compositions. In one embodiment, the composition is pharmaceutical composition, and the carrier is pharmaceutically acceptable carrier. In one embodiment, the composition is cosmeceutical composition, and the carrier is cosmeceutically acceptable carrier.

According to some embodiments of the above composition, triazelaine is the carrier. In the context of the invention, when triazelaine is the carrier designed to deliver at least one biomolecule and/or biological sample to its target.

According to some embodiments of the above composition, the TriAza carrier fulfills the function of a preservative to prevent bacterial and yeast contamination.

According to some embodiments of the above composition, the non-limiting list of biomolecules includes deoxyribonucleic acids (DNA), ribonucleic acids (RNA), organic molecules, inorganic molecules, amino acids, vitamins, polyphenols, steroids, peptides, polypeptides, and protein complexes.

According to some embodiments of the above composition, the sample of microbiome is donor-autologous microbiome, allogenic microbiome, or in-vitro cultured microbiome.

According to some embodiments of the above composition, the non-limiting list of active agents includes anti-microbial agent, anti-aging agent, anti-viral agent, anti-fungal agent, anti-bacterial agent, antioxidant, anti-inflammatory agent, antibiotics, anti-parasitic agent, anesthetic agent, analgesic agent, anti-allergic agent, antipruritic agent, immunosuppressant, anti-angiogenic agent, vasoconstrictors, and a probiotic agent.

According to some embodiments of the above composition, the one or more active agent is a sample of non-lipase probiotic microorganisms. According to the embodiments of the invention, the list of non-lipase probiotic microorganisms includes, without limitation, non-lipase probiotic strains of microorganisms known to be bio-protective to the skin, Bifidobacterium (Bif. Adolescentis, Bif. Animalis, Bif. Breve, Bif. Infantis, Bif. Longum, Bif. Thermophilum), Lactobacillus (Lact. Rhamnosus, Lact. Salivarius, Lact. Acidophilus, Lact. Brevis, Lact. Casei, Lact. Curvatus, Lact. Fermentum, Lact. Gasseri, Lact. Johnsonii, Lact. Reuteri), Streptococcus (Strep. Thermophilus), Enterococcus (Ent. Faecium), Lactococcus (L. lactis subsp. cremoris, L. lactis subsp. lactis).

According to some embodiments of the above composition, the composition may be in the form powder, suspension, emulsion, cream, paste, gel, ointment, spray, foam, soap, shampoo, and oil.

According to some embodiments, pH of the composition of the invention is from 5 to 8. According to some embodiments, the pH of the composition is 5, 5.5, 6, 6.5, 7, 7.5, and 8.

According to some embodiments, the above composition further comprises a surfactant, a preservative, a colorant, or any combination thereof.

According to some embodiments, triazelaine content in the above composition is from 1% to 100%. According to some embodiments, triazelaine content in the above composition is 5%-to 95%; 10% to 80%; 15% to 70%; 20% to 60%; and 30% to 50%. According to some embodiments, triazelaine content in the above composition is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 46%, 47%, 48%, 49%, 50%, 51%, 52%, 53%, 54%, 55%, 43%, 44%, 45%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%.

According to some embodiments, the invention provides a topical composition according to the above embodiments, for use in the treatment of skin condition associated with a lipase-producing microorganism. A non-limiting list of skin conditions associated with a lipase-producing microorganism includes acne, rosacea, atopic dermatitis, seborrhea, abnormal pigmentation including melasma, aging, eczema, or any other disease or condition associated with lipase-producing microorganism. A non-limiting list of lipase-producing microorganism includes lipophilic bacteria species such as Pseudomonas spp, Cutibacterium acnes, Staphylococcus, including S. aureus and S. Hominis and Corynebacterium, including acne pathogen, also fungi species such as Candida, including C. Albicans, such as Acinetobacter, such as A. Radioresistens, yeast, such as Malassezia.

According to some embodiments, the invention provides a topical composition according to the above embodiments, for use in the treatment of conditions such as eczema, acne, atopic dermatitis, seborrheic dermatitis, tissue necrosis, skin sores, psoriasis, cellulitis, fungal infection, lesion, wound, disorder of the pilosebaceous unit, shaving and contact induced skin irritation, wounds.

According to some embodiments, the invention provides a composition according to one or more of the above embodiments for use as a medicament. According to some embodiments, the composition is a topical composition.

According to some embodiments, the invention provides a topical composition comprising a prodrug having the formula:

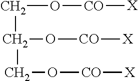

wherein X is azelaic acid, and wherein upon exposure to a bacterial lipase, azelaic acid is cleaved to become released from said compound.

According to some embodiments, the invention provides a topical composition for the treatment of a skin disorder comprising an active therapeutic agent and triazelaine as a carrier.

According to some embodiments, the invention provides a topical composition for skincare comprising triazelaine. In one embodiment, the composition further comprises at least one biomolecule. In one embodiment, the composition further comprises a sample of microbiome.

According to some embodiments, the invention provides a composition according to one or more of the above embodiments for use in the treatment of a disease or condition. According to some embodiments, the composition is a topical composition and the disease or condition is an inflammation of the skin.

According to some embodiments, the invention provides topical compositions for the treatment of lipase virulent factor overexpressed pathogenic microbiome such compositions preferably utilizing as active ingredient azelate esters, such as tri-azelate glycerol, alone, or in combination with other active ingredients.

According to some embodiments, the invention provides a method of treating an inflammatory condition of the skin in a subject in need of such treatment, comprising topically administering to the subject an amount of triazelaine effective to treat said condition.

According to some embodiments, the invention provides a method of slowing and/or preventing progression of an inflammatory condition of the skin in a subject, comprising administering to the subject the topical composition according to the above embodiments.

According to some embodiments, the invention provides topical anaplerotic compositions. As used herein, the term “anaplerotic composition” refers, without limitation, to composition triggering anaplerosis (or anaplerotic reactions). Anaplerosis is defined as the regenerative metabolic reaction aimed for the replenishment of intermediates of tricarboxylic acid cycle (TCA cycle).

According to some embodiments, the above and foregoing topical anaplerotic compositions are suitable for the treatment of a variety of skin abnormalities associated, without limitation, with inflammation, microbiome disbalance and/or aging.

According to some embodiments, the above and foregoing topical anaplerotic compositions are based on MOCFA, precursors and derivatives thereof. In one embodiment, the above compositions are based on C9 MOCFA.

According to some embodiments, the above and foregoing topical anaplerotic compositions are based on the combination medium odd chain lipids, preferably C9 MOCT/MOCFA and derivatives thereof.

According to some embodiments, the above and foregoing compositions comprise Triolein in carrier oil and (ii) as an active anaplerotic ingredient-MOCT-the TG and fatty acid moieties, each with 9 carbon atoms (C9 MOCTA/OCFA). In some embodiments the acids are nonanoic carboxylic acid and/or dicarboxylic nonanoic fatty acids or derivatives of thereof.

According to some embodiments, the above and foregoing compositions formulated as C9 MOCTA/OCFA mixture of aliphatic esters such as, without limitation, fatty acid ethyl esters, triglycerides (TG) triacylglycerols (TAG), diacyl glycerols (DAG), monoacyl glycerols (MAG), and derivatives thereof.

According to some embodiments, the above and foregoing compositions are stable for at least 6 months, preferably 10 months, most preferably 12 months or greater than 12 months. In some embodiments, the compositions are stable for 24 months. In some embodiments, the composition comprises high concentration of carrier oils rich with endogenous inhibitors of Free Radicals and antioxidants, such as, without limitation, lipophilic antioxidants, vitamin E, tocopherols, carotenoids, and phenols In addition, tocopherols.

According to some embodiments, the above and foregoing compositions further comprise at least one active agent suitable for promoting antiaging functionality or sustaining the aging process. A non-limiting list of agents includes antioxidant; ROS/FR inhibitors, chelators, vitamin E and derivatives, vitamin C and derivatives, FA transporters, carnitine, Krebs cycle/TCA substrates, metabolic modulators, glucose modulator-2DG such as 2 deoxyglucose, ketogenic substrates, MCT, coQ10, ATP modulators and derivatives, nicotinamide derivatives, nicotinamide adenine dinucleotide (NAD), nicotinamide riboside, nicotinamide mononucleotide, SIRT modulators, resveratrol, quercetin, pterostilbene, oleic acid, autophagy regulators, polyamines, mTOR inhibitors, caffein, fisetin, EGCG, telomere modulators, skin lightening/depigmentation agents, kojic acid, retinol, emollients, probiotics, prebiotics, postbiotics, nucleic acids stabilizers, DNA stabilizers, RNA stabilizers, spermidine, epigenetic modulators, Histone deacetylases (HDACs), short chain fatty acids, butyric acid derivatives, histone methyl transferases (HMTs), DNA methylation inhibitors, stilbens, hormone replacement agents, estrogen, DHEA, testosterone, oxytocin, polyphenols, phytosterols, steroids, flavonoids, inflammation/AHR inhibitors and indoles.

According to some embodiments of the above and foregoing compositions, the concentration of the at least one active agent suitable for promoting antiaging functionality or sustaining the aging process is at least 1%, preferable 2, 5%, most preferable 5% or greater. In some embodiments, at least 50% of the activity of the at least one active agent suitable for promoting antiaging functionality or sustaining the aging process is impacted/affected by the endogenous properties of selected carrier oil composition. In the context of the invention, the phrase “impacted/affected by the endogenous properties of selected carrier oil composition” is meant to be understood as positive, protective effect on functional molecular mechanisms that prevent or treat inflammation or aging molecular processes.

According to some embodiments of the above and foregoing compositions, the carrier oil constitutes 50% to 90% of the composition. In some embodiments, the carrier oil constitutes 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% of the composition. In some embodiments the oil carrier TG is Triolein.

According to some embodiments of the above and foregoing compositions, C9 MOCTA/OCFA constitutes between 5% to 25% of said composition. In some embodiments, the C9 MOCTA/OCFA constitutes 5%, 6%, 7%, 8%, 9%, 10%, 11%, 12%, 13%, 14%, 15%, 16%, 17%, 18%, 19%, 20%, 21%, 22%, 23%, 24%, 25%.

According to some embodiments of the above and foregoing compositions, at least one active agent suitable for promoting antiaging functionality or sustaining the aging process, may constitute 5% to 25% of said composition. In some embodiments, at least one active agent suitable for promoting antiaging functionality constitutes 5%, 6%, 7%, 8%, 9%, 10%, 11%, 12%, 13%, 14%, 15%, 16%, 17%, 18%, 19%, 20%, 21%, 22%, 23%, 24%, 25%.

According to some embodiments, the above and foregoing anaplerotic compositions comprise: a first component selected from triglycerol, diglycerol, monoglycerol of pelargonic acid, triacylglycerol of Azelaic acid, or a mixture of thereof; and a second component selected from azelaic acid, pelargonic acid, and linear, branched esters and diesters thereof.

According to some embodiments of the above and foregoing anaplerotic compositions, the mol/mol ratio of MOCT-C9 to MOCFA-C9 is from 10:90 to 90:10.

According to some embodiments of the above and foregoing anaplerotic compositions, the combined amount of MOCT-C9 and MOCFA-C9 make up at least 5 mol % of the lipids in the composition.

According to some embodiments of the above and foregoing anaplerotic compositions, the mol/mol ratio of MOCT-C9 to MOCFA-C9 is 20:80 to 85:15; 20:80 to 80:20; 30:70 to 85:15; 40:60 to 75:25; 50:50 to 70:30; 50:50 to 67:33; 55:45 to 65:35; 58:42 to 62: 38.

According to some embodiments of the above and foregoing anaplerotic compositions, the total amount of triglycerides and fatty acids corresponds to: (i) C9 precursors and derivatives, such as, without limitation, pelargonic acid, azelaic acid, nonanoic esters; triglycerides such as, without limitation, Triheptanoin, Tripelargonine, and TriAza) and (ii) lipids of carrier oil. In some embodiments, the lipids of carrier oil are in the amount of at least about 30%, more preferably about 40%, most preferably about 50% or greater of total lipids of said compositions.

According to some embodiments, the invention provides topical anaplerotic compositions based on C9 MOCFA and derivatives thereof for the treatment of skin abnormalities associated with aterations in sebaceous microbiome. A non-limiting list of abnormalities associated with aterations in sebaceous microbiome includes redness, irritation, acne, seborrheic disorder, eczema, atopic dermatitis inflammatory symptoms, overdryness or oilness of the skin, non-sooth/even surface or abnormal pores, uneven or hyperpigmentation, itching, loss of skin integrity, wrinkles-“aged appearance”.

According to some embodiments, the invention provides topical anaplerotic compositions based on C9 MOCFA and derivatives thereof for the treatment of skin abnormalities associated mitochondrial dysfunction.

According to some embodiments, the invention provides topical anaplerotic compositions based on C9 MOCFA and derivatives thereof for preventing skin abnormalities associated impaired lipogenesis and mitochondrial dysfunction.

According to some embodiments, the invention provides topical anaplerotic Triglecyride (TG) lipids-based and fatty acids (FA) precoursors enriched compositions for preventing and/or improving and/or trating and/or repairing impaired lipidome and mitichondrial dysfuntion.

According to some embodiments, the invention provides anaplerotic topical compositions comprising Triolein in carrier oil and a combination of Medium Odd Chain Tryglicerydes (MOCT) with MOCFA.

According to some embodiments the above and foregoing compositions provide improved penetration of C9 FAs into the adult human skin.

According to some embodiments, the invention provides a sebum replacement therapy based on active anaplerotuc compositions.

According to some embodiments, the invention provides compositions comprising TriAza for the treatment of skin inflammation, skin disorders associated with activity of lipases secreted by microorganisms, and impaired microbiome.

According to some embodiments, the invention provides a method of treating and/or preventing and/or slowing progression of conditions associated with inflammation, microbiome disbalance and/or aging.

According to some embodiments, the above method comprises administering to a subject in need an affective amount of topical anaplerotic compositions according to one or more of the above embodiments.

According to some embodiments of the above methods and compositions, triozonoids can be used as a precursor of TriAza and PA, that in situ will be oxidized to TriAza by acidic pH, oxygen, Zn, or hydrogen peroxides, or other hydroxyl or superoxide donors to decompose ozonoides to carboxylic aliphatic derivatives, such as TriAza.

According to some embodiments of the above methods and compositions, the non-limiting list of C9 FAs includes: Pelargonic Acid-PA, also called nonanoic acid FA-Fatty Acid, a esters of PA and branched PA-isononanoic acid including list of potential ingredients, such as Pentaerythrityl Tetrapelargonate; Diethylene Glycol Diethylhexanoate/Diisononanoate, Ethyl Pelargonate; Diethylene Glycol Diethylhexanoate/Diisononanoate; Butylene Glycol Diisononanoate, Cetearyl Isononanoate, Cetearyl Nonanoate, Cetyl Isononanoate, Diethylene Glycol Diethylhexanoate/Diisononanoate, Dipentaerythrityl Pentaisononanoate, Ethylhexyl Isononanoate, Isodecyl Isononanoate, Isononyl Isononanoate, Isostearyl Isononanoate, Isotridecyl Isononanoate, Neopentyl Glycol Diisononanoate, Tridecyl Isononanoate, Ethylhexyl Pelargonate, Ethyl Pelargonate, Isobutyl Pelargonate, Methyl Pelargonate; Butylene Glycol Diisononanoate, Pentaerythrityl Tetraisononanoate, Propylene Glycol Diisononanoate, Pentaerythrityl Tetrapelargonate; Pelargonic Acid, Polyglyceryl-20 Octaisononanoate; Butylene Glycol Diisononanoate, Dipentaerythrityl Pentaisononanoate, Pentaerythrityl Tetrisononanoate, Propylene Glycol Diisononanoate, Pentaerythrityl Tetrapelargonate, (E,Z)-2,6-nonadienal; trans-2, cis-6-nonadienal; Heptanoic acid, ester with 2,2-dimethyl-1,3-propanediol; Ethyl pelargonate; nonanal-C9 aldehyde. In addition, glycerol esters of C9-are mono-di- and triglycerides of nanonoic acid-Tripelargonin, Cetearyl Isononanoate, Glycerin, Isononyl Isononanoate.

According to some embodiments of the above methods and compositions, the non-limiting list of TGs includes: heptanoic acid and esters of thereof including Triheptanoin TG7, Dicarboxylic C9 azelaic acid and salts and di-and monoesters; glycerol esters, mono-di and Triazelaine; Tripelargonin, Triazelaine, Triheptanoin, analogues of C9 glycerol esters and dicarboxylic azelaic acid.

According to some embodiments of the above methods and compositions, oil enriched with Triolein is used as sebum antiaging replacement therapy. In some embodiments, the oil is characterized by comedogenic index of 0-2 max (out of 5 grades of comedogenicity). The non-limiting list of oils includes argan oil, apricot kernel oil, rose hip oil, Simmondsia Chinensis (Jojoba) Seed Oil, rose flower oil (Rosa Damascena Flower Oil), Olea Europaea (Olive) Fruit Oil, most preferably olive oil BP certified, castor oil, coconut oil, soybean oil, canola oil, rapeseed oil, corn oil, cottonseed oil, peanut oil, safflower oil, sesame oil, sunflower oil (Helianthus Annuus (Sunflower) Seed Oil), linseed oil, palm kernel oil, tung oil, jatropha oil, camelina oil, pennycress oil, wheatgerm oil, pistachio oil, poppy oil, pine oil, Persea Gratissima (Avocado) Oil, hazel nut oil, grapeseed oil, colza oil, cade oil, peach kernel oil, coffee bean oil, Michelia Alba Leaf Oil, Silybum Marianum Seed Oil, Camellia Japonica Seed Oil, Carnosine, and palm oil. In some embodiments, the carrier oil is loaded with oleic acid in an amount of at least to 30% of total content of carrier oil and said oleic acid in carrier oil has functional activity in improving penetration of active ingredients into skin layer so will be bioavailable in epidermis and dermis to enter intracellular and provide desired functionalities. In some embodiments, the carrier oil comprises endogenous antioxidants preferably vit E-tocopherol, and phenols.

As used herein, the phrase “slowing and/or preventing progression” refers, without limitation, to the influence of the treatment on the clinical course of the disease or the condition. For example, in the case of acne, illness severity ranges from mild to severe, while mild disease is categorized as <30 total lesions count to moderate disease is categorized as 30-125 of total lesions count; severe disease has manifestations of >125 of total lesions count. In the context of the invention, the proposed therapy is aimed at slowing and/or preventing the transition from mild to severe illness. The “slowing and/or preventing” progression of the condition according to the embodiments of the above method may be measured using any appropriate questionary, method, scale, diagnostic tool, or any other means that are known in the art or acceptable by the relevant functions and professionals. The term “preventing” might, but does not necessarily, means recovery from the illness. As such, the term “preventing” relates to the situation when the patient does not present symptoms and/or signs and/or manifestations of the next “stage” of illness severity as defined by the appropriate and acceptable parameters for the specific disease condition. The term “slowing”, or attenuating can, without limitation, prolong the time of transition into the next “stage” of illness severity, thus providing greater window of opportunity for extensive care and recovery.

According to some embodiments, the invention provides a method of treating an inflammatory condition of the skin in a subject, comprising administering to the subject the topical composition according to the above embodiments. In one embodiment, the inflammatory condition of the skin is associated with a lipase-producing microorganism, including, without limitation, Pseudomonas spp., Cutibacterium acnes, Staphylococcus, and Corynebacterium.

According to some embodiments, the invention provides a method of treating an inflammatory condition of the skin such as, without limitation, eczema, acne, atopic dermatitis, tissue necrosis, skin sores, psoriasis, cellulitis, fungal infection, gangrene, and disorder of the pilosebaceous unit, comprising administering to the subject the topical composition according to the above embodiments.

According to some embodiments, the invention provides a method of treating localized skin inflammation comprising administering to the site of the inflammation the topical composition according to the above embodiments. In the context of the invention, the term “localized skin inflammation” is meant to be understood as local inflammatory response, which is limited to a certain, well-defined area/portion/region of the skin affected by the harmful stimulus. The localized skin inflammation according to the embodiments of the invention may appear simultaneously in several parts of the body.

According to some embodiments, the invention provides a method of balancing the microbiome of the skin in a subject in need comprising administering to the subject the topical composition according to the above embodiments. In the context of the invention, the term “balancing the microbiome” is meant to be understood as maintaining the possibility of microbiome homeostasis to control by selectively targeting target the desired opportunistic or/and pathogenic species of microbiome which causes the production of the triglyceride lipase as a virulent factor causing disease or disorder and thus reaching desired effect in reduction of disease or disorders symptoms while keeping microbiome balanced.

According to some embodiments, the invention provides a method of treating a condition associated with disbalance of skin microbiome in a subject in need comprising administering to the subject the topical composition according to the above embodiments.

According to some embodiments, the invention provides a method of maintaining the balance of skin microbiome in a subject in need comprising administering to the subject the topical composition according to the above embodiments.

According to some embodiments, the invention provides a method of preventing aging of the skin in a subject in need comprising administering to the subject the topical composition according to the above embodiments. Classical manifestations of aging include genomic instability and telomere attrition, epigenetic alterations, and loss of proteostasis, deregulated nutrient-sensing, mitochondrial damage and dysfunction, cellular senescence, stem cell exhaustion/dysregulation, and altered intercellular communication. In the context of the invention, the term “aging of the skin” is interchangeable with such terms as “skin abnormalities/diseases/disorders associated with aging”, and refers, without limitation, to phenomena such as decline of both absolute and relative amounts of sebaceous TG, skin dryness, lack of brightness, loss of skin microbiome, wrinkling, loss of elasticity, laxity, rough-textured appearance of the skin, sebaceous glands (SG) senescence and SG atrophy, and disturbance of skin barrier. The aging process is usually accompanied by mitochondrial dysfunction, phenotypic changes in cutaneous cells as well as structural and functional changes in extracellular matrix components such as collagens and elastin.

The present invention further provides a lipase-hydrolyzable (lipase-mediated) site-targeting sytem releasing upon hydrolysis an inert, or non-inert, carrier compound (referred to as “carrier component”) and an active compound (referred to as “active ingredient”), selected from the group consisting of:

According to some embodiments, a lipase-hydrolyzable triglyceride ester comprises an inert carrier component (glycerol moiety) and an active component (fatty acid moiety). For example, a component, such as azelaic and/or retinoic acid is chemically attached to the glycerol backbone to form tri-retinoic, tri-azelaic and mixed di-retinoic, mono-azelaic, or di-azelaic, mono-retinoic ester derivatives.

The triglyceride ester of the present invention provides three molecules of active fatty acids per one mole of ester.

According to some embodiments, a lipase-hydrolyzable moiety comprising an antibiotic component (such as erythromycin, macrolide or lincosamide moiety, natural antimicrobial preservative, e. g. alfa-pinene) and an anti-acne active carrier (such as linoleic or oleic, azelaic diester acid moiety).

According to some embodiments, ester of the present invention provides one molecule of anti-acne active fatty acid (serves also as a carrier component) and an antibiotic material per one mole of ester.

According to some embodiments, the invention provides a compound comprising a moiety of an inert cholesterol or cholesterol-like carrier and a moiety of an antibiotic active ingredient (such as macrolide, preferably 14-membered macrolide, or lincosamide, preferably clindamycin).

According to some embodiments, the invention provides methods for selective depletion of virulent pathologic microbiome and enhancement of protective microbiome by preserving and controlling the risk of contamination by pathogenic species. Disclosed selective methods and compositions useful for treatment/control of lipase-producing pathogenic/virulent microbiome without damaging major host protective microbiome.

According to some embodiments, the invention provides method and compositions useful for treating acne-related disorders, lipase producing, virulent/pathogenic microorganisms—an important target for the disclosed composition.

According to some embodiments, the disclosed compositions are useful for the treatment of S. aureus infections, including, without limitation those, produced by methicillin-MRSA and vancomycin-resistant strains VRSA.

According to some embodiments, the disclosed compositions can be used alone or in combination with traditional antimicrobials and antibiotics to treat such and can be used in settings such as, without limitation, foreign-body, catheter or endovascular infections, hospital acquired or post-operative infections, recurrent skin infections, or, in case of S. aureus infections in an immunocompromised host.

According to some embodiments, the disclosed compositions can serve as preservative agent to prevent contamination and maintain sterility of topical formulations, such as, without limitation, prevention of Pseudomonas, Candida, Staphylococcus, and more (specifically species are restricted by USP and European Pharmacopoeia for release of cosmetic, topical dermatologic and other formulations and compositions).

According to some embodiments, the invention provides use of disclosed compositions in combination with “green” or natural surfactants. In the context of the invention, the term “green” surfactant is defined as a substance obtainable from natural and/or sustainable and/or renewable sources, such as, without limitation, glutamate, lauric acid, vitamin E, succinic acid, lactic acid, alkyl polyglycosides, alkyl glucoside, sodium coco sulfate (sodium lauryl sulfate). The green, nonionic surfactants of the disclosed compositions include, without limitation, sugar-based surfactants, polyol-based surfactants, alkyl ethers, and alkyl carbonates. The sugar-based surfactants include, without limitation, alkyl polyglycoside (or alkyl polyglucoside) surfactants made from fatty alcohols in coconut oil and polyglucose in corn. In addition to its excellent ecological profile, alkyl polyglycosides are biodegradable, and do not irritate human skin. Additional nonionic green surfactants suitable for use in the disclosed composition may include, but are not limited to, alkyl glucose amide, triglycerides, N-methyl coconut fatty acid glucamides (C12-14), amino acid-based surfactants, sugar esters, sorbital esters, sterol esters, glycolipid biosurfactants, etc. The green anionic surfactants may also be prepared from immediate precursors that are obtainable from natural and renewable sources. Accordingly, the green anionic surfactants may include one or more long-chain alkyl sulfates. Suitable green anionic surfactants include, without limitation, sodium coco sulfate or sodium lauryl sulfate. Sodium coco sulfate may be prepared from sulfating coconut oil, which is made up of a wide range of fatty acids (ranging from as few as 8 carbon alkyl chains to as many as 20, 45-50%, of the fatty acids in coconut oil are fatty acids containing 12 carbons).

In addition, PEG derived “green” vitamin E-derived surfactant can also be derivative of Vitamin E TPGS, including Vitamin E TPGS 200, Vitamin E TPGS 300, Vitamin E TPGS 400, Vitamin E TPGS 1000, Vitamin E TPGS 1500, Vitamin E TPGS 2000 and Vitamin E TPGS 4000. Natural derived green surfactants can belong to the group of the polyglyceryl surfactants and can be selected, without limitation, from polyglyceryl monoesters or polyglyceryl multi-esters. Non-limiting examples of polyglyceryl monoesters contemplated herein include polyglyceryl-4-caprate, polyglyceryl-4-caprylate, polyglyceryl-4-laurate, polyglyceryl-4-isostearate, polyglyceryl-4-oleate, polyglyceryl-5-laurate, polyglyceryl-5-myristate, polyglyceryl-5-isostearate, polyglyceryl-5-oleate, polyglyceryl-5-stearate, polyglyceryl-6-isostearate, polyglyceryl-6-oleate, polyglyceryl-6-stearate, polyglyceryl-8-oleate polyglyceryl-8-stearate, polyglyceryl-10-laurate, polyglyceryl-10-myristate, polyglyceryl-10-palmitate, polyglyceryl-10-isostearate, polyglyceryl-10-linoleate, polyglyceryl-10-oleate, polyglyceryl-10-stearate, polyglyceryl-10-behenate/eicosadioate, polyglyceryl-10-hydroxystearate/stearate/eicosadioate, and/or polyglyceryl-10-fatty ester (POLYALDO.RTM. 10-2-P). Non-limiting examples of multi-esters contemplated in the composition polyglyceryl disclosed herein include polyglyceryl-5-triisostearate, polyglyceryl-5-dioleate, polyglyceryl-5-trioleate, polyglyceryl-6-tricaprylate, polyglyceryl-6-dioleate, polyglyceryl-6-distearate, polyglyceryl-6-pentastearate, polyglyceryl-6-octastearate, polyglyceryl-8-decaerucate/decaisostearate/decaricinoleate, polyglyceryl-10-caprylate/caprate, polyglyceryl-10-dipalmitate, polyglyceryl-10-diisostearate, polyglyceryl-10-pentaisostearate, polyglyceryl-10-nonaisostearate, polyglyceryl-10-decaisostearate, polyglyceryl-10-dioleate, polyglyceryl-10-pentaoleate, polyglyceryl-10-decaoleate, polyglyceryl-10-distearate, polyglyceryl-10-tristearate, polyglyceryl-10-pentastearate, polyglyceryl-10-pentahydroxystearate, and polyglyceryl-10-heptahydroxystearate. Water-soluble salts of C.sub.8-20 alkyl sulfates, sulfonated monoglycerides of C.sub.8-20 fatty acids, sarcosinates, taurates and the like. Exemplary embodiments of these and other classes include, without limitation, sodium lauryl sulfate, sodium lauryl ether sulfate, ammonium lauryl sulfate, ammonium lauryl ether sulfate, sodium cocoyl monoglyceride sulfonate, sodium lauryl sarcosinate, sodium lauryl isethionate, sodium laureth carboxylate, and sodium dodecyl benzenesulfonate. In some embodiments, the anionic surfactant is sodium lauryl sulfate (SLS). Co-surfactants include naturally derived lecithins, e.g. sunflower lecithin.

According to some embodiments, the invention provides use of disclosed compositions as a delivery vehicle to improve permeability, to control the release (by skin microbiome) of azelaic acid component as an active compound to reduce of irritation and low permeability of pure azelaic acid.

According to some embodiments, the invention provides specific compositions and additives of TriAza-based microbiome biocontrol delivery system of prebiotic, post biotic and probiotics.

According to some embodiments, the invention provides application of TriAza as a delivery and microbiome-stabilizing reagent for probiotic, prebiotic, postbiotic and microbiome compositions topical applications.

According to some embodiments, the invention provides selective compositions for enhancement of probiotic strains of microorganisms to the skin, such as, without limitation, Bifidobacterium (Bif. Adolescentis, Bif. Animalis, Bif. Breve, Bif. Infantis, Bif. Longum, Bif. Thermophilum), Lactobacillus (Lact. Rhamnosus, Lact. Salivarius, Lact. Acidophilus, Lact. Brevis, Lact. Casei, Lact. Curvatus, Lact. Fermentum, Lact. Gasseri, Lact. Johnsonii, Lact. Reuteri), Streptococcus (Strep. Thermophilus), Enterococcus (Ent. Faecium), Lactococcus (L. lactis subsp. cremoris, L. lactis subsp. lactis).

According to some embodiments, the invention provides use of TriAza as a selective antimicrobial delivery vehicle or preservative to deliver non-lipase probiotic strains of microorganisms known to be bio-protective to the skin, such as, without limitation, Bifidobacterium (Bif. Adolescentis, Bif. Animalis, Bif. Breve, Bif. Infantis, Bif. Longum, Bif. Thermophilum), Lactobacillus (Lact. Rhamnosus, Lact. Salivarius, Lact. Acidophilus, Lact. Brevis, Lact. Casei, Lact. Curvatus, Lact. Fermentum, Lact. Gasseri, Lact. Johnsonii, Lact. Reuteri), Streptococcus (Strep. Thermophilus), Enterococcus (Ent. Faecium), Lactococcus (L. lactis subsp. cremoris, L. lactis subsp. lactis).

According to some embodiments, the invention provides TriAza formulated with postbiotic composition. An example of such postbiotic is, without limitation, a secondary bile salt and/or acid metabolites, while Bacteroides and lactobacillus species metabolize such metabolites. Accordingly, postbiotic may be presented by Bifidobacterium-processed sphingolipids. Such postbiotic may be delivered directly or indirectly as a combo of Bifidobacterium or/and Lactobacillus delivered by TriAza based formulation and relevant substrate enrichment (i.e. bile acids, sphingolipids, etc.). Accordingly, it can result in metabolites, such as dihydroceramides, and oxidated, hydroxylated and other derivatives/metabolites of cholic acids.

According to some embodiments, the invention provides active agents and methods of treating rosacea.

According to some embodiments, TriAza can be used in combinations with anti-rosacea agents, such as, without limitation, topical alpha-adrenergic receptor agonists such as brimonidine and oxymetazoline; nonselective beta-blockers, botulinum toxin, topical sodium sulfacetamide, topical metronidazole, topical ivermectin, topical retinoids, topical calcineurin inhibitors, and more. According to some embodiments, the above combinations demonstrate a synergistic effect in anti-rosacea treatment.

According to some embodiments, TriAza-based compositions are used as an anti-infective agent, anti-seborrheic, anti-acne, ands anti-candida, as a preservative and as a deodorant.

According to some embodiments, the invention provides natural-based preservative in combination with other preservatives to reduce toxic concentrations and get complementary, additive effect on product stability.

According to some embodiments, the invention provides TriAza-based cosmetic preparations.

According to some embodiments, the invention provides TriAza-based composition comprising active ingredients that enhance Azelaic acid functionality.

According to some embodiments, the invention provides TriAza based composition that can be used as active ingredient and/or as a diluent.

According to some embodiments, the invention provides TriAza that can be used as a cosmetically acceptable diluent “base” with preservative activity.

According to some embodiments, TriAza based composition can serve as a cosmetically acceptable base, and/or as an active bacteriostatic antiseptic and antibiotic composition, as preservative, lightening agent, azelaic acid prodrug for approved medical and cosmetic indications, and can be mixed with other functional actives or can be added to accepted formulations.

According to some embodiments, the cosmetically acceptable base acts as a diluent, dispersant and/or a carrier for other materials present in the composition and can optionally facilitate their distribution when the composition is applied to the skin.

According to some embodiments, a non-limiting list of cosmetic active agents that can be used with Azelaic acid includes: vitamin B6, vitamin C, A, resorcinol vitamin derivatives, 12-hydroxystearic acid, glutathione precursors, galardin, adapalene, aloe extract, ammonium lactate, arbutin, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, gluco pyranosyl-1-ascorbate, gluconic acid, glycolic acid, green tea extract, 4-Hydroxy-5-methyl-3 [2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, octadecenedioic acid, acetyiglucosamine, pitera extract, symwhite, calcium pantothenate (Melano-block), seppiwhite, soybean extract (bowman birk inhibitor) and mixtures thereof, vitamin B6, resorcinol derivatives like e.g. 2,4-substituted resorcinol derivatives and 3,5-substituted resorcinol derivatives, hexylresorcinol and phenylethyl resorcinol, 12-hydroxystearic acid, glutathione precursors, galardin, beta.-alanine derivatives including 1-Piperidinepropinoic (1PP) acid, as wrinkle improving agent, steroidal anti-inflammatory agents, non-steroidal anti-inflammatory agents, local anesthetic, antiangiogenic agents, derivatives of retinoic acid, natural compounds, and compounds that act as sunscreens, skin humectants, antioxidants such as e natural phenols, flavonoids, lycopene, terpenes, stilbens, cannabinoids, essential oils, phytosterols and telomerase activators plant based extracts (TA 65, etc.), medicinal mushroom extracts (Reishi, Ganoderma, Cordycepts), adaptogens, anti-aging compounds, plant and medicinal mushroom extracts, sterols, ascorbic acid and its esters, sodium bisulfite, butylated hydroxytoluene, butylated hydroxyanisole, tocopherols, and chelating agents like EDTA, succinic acid and citric acid.

According to some embodiments, the invention provides topical, cosmetic, and dermatological formulations preparation and inactive ingredients, excipients, and enhancers.

According to some embodiments, a non-limiting list of pharmaceutically acceptable excipients includes protectives, adsorbents; pharmaceutically acceptable carrier is selected from the group including sprays, mists, aerosols, solutions, lotions, gels, creams, ointments, pastes, unguents, emulsions, and suspensions. Optional excipients are demulcents, emollients, preservatives, antioxidants, moisturizers, buffering agents, solubilizing agents, skin-penetration agents, and surfactants.

According to some embodiments, selected additional functional agents may present in an amount in the range of about 0.01 to 5 weight percent, wherein the said TriAza based topical composition having a physiologically acceptable pH in the range of about 5 to 8.

According to some embodiments, the excipients added to maintain the osmolality are in the range of 250-650 mOsmol/kg.

According to some embodiments, the cosmetically acceptable base may be made of ingredients that include, without limitation, fatty acids having from 10 to 30 carbon atoms and salts thereof, water, liquid or solid emollients, solvents, humectants, thickeners, powders. These ingredients may be used alone and/or in combination with each other to form the cosmetically acceptable base.

According to some embodiments, the cosmetically acceptable base may contain skin penetration enhancers such as, without limitation, dimethyl sulfoxide.

According to some embodiments, emollients that may be used in the cosmetically acceptable base include, without limitation, stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl isocetyl alcohol, eicosanyl alcohol, oleate, octadecan-2-ol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate, ethyl alcohol, isopropanol, acetone, ethylene glycol monoethyl ether, diethylene glycol monobutyl ether, diethylene glycol monoethyl ether, and mixtures thereof.

According to some embodiments, topical TrAza formulations have a pH buffers in the range of 6.5-7.0 such as, without limitation, phosphate buffer, citrate buffer, and acetate buffer.

According to some embodiments, the compositions of the invention are stable and comparable to the control for six months of storage at 25° C.

According to some embodiments, TriAza-based soap produced by catalytic ozonolysis and subsequent purification step is stable (without change of odor, color, and wax-like consistency)—for over two years at 25° C. in light.

According to some embodiments, TriAza was prepared based on hydrogen peroxide/permanganate oxidation of crude olive oil. Non-limiting list of natural additives that were added to various fractions includes: probiotic mixture, extracts, essential oils, postbiotic, and prebiotics.

According to some embodiments, the invention provides compositions and use of TriAza as a functional formulation.

According to some embodiments, the composition may include, without limitation, amphiphilic biomaterial and antimicrobial system that may incorporate active compounds to the skin.

According to some embodiments, TriAza may be useful in decreasing concentration of antimicrobial agents required to achieve an effective reduction in opportunistic pathogenic microorganisms that typically infect wounds, as well as formation of biofilms and hospital acquired infections thus reducing risk of resistance.

According to some embodiments, TriAza combined with conventional antibiotics and antifungal active agents, and/or with nicotinamide natural anti-planktonic and/or anti-Candida active results in significant reduction of MIC of the active ingredients.

According to some embodiments, TriAza combined with conventional antibiotics and antifungal active agents, and/or with nicotinamide natural anti-planktonic and/or anti-Candida leads to the reduction of the required dosage, simplifies formulation, improves compliance of administration, reduces risk of resistance, and side effects.

According to some embodiments, TriAza-based soaps are an effective treatment option against infection-associated conditions induced by P. acnes; S. aureus and C. albicanas that also relevant to Pseudofolliculitis Barbae (PFB) dermatological post-shaving disorder.

According to some embodiments, minimum inhibitory concentrations (MIC) and minimum bactericidal concentrations (MBC) of TriAza and other combinations on S. aureus and P. aeruginosa are determined using any standard method, such as, without limitation, a protocol according to which overnight cultures of S. aureus and P. aeruginosa are grown in TSB and adjusted to 0.1 optical density (O.D.); Each culture is then incubated with two-fold serial dilutions of tested TriAza or combination.

According to some embodiments, TriAza based compositions can be tested for additive and synergistic antimicrobial properties with other microbiome-controlling agents shown to prevent formation of biofilm by resistant strains.

According to some embodiments, disclosed combinations of Tri-Aza with L-Glu acetic amino acid in both bacteria that can be applied in multiple indications at risk of antibiotic resistance.

According to some embodiments, additional natural antibiotics may be used in risk patients as additive, adjunctive, or preventive method, for example, without limitation, plant extracts, tea tree oil, myrth oil, rose oil, limonene, pinene, other essential oils, copper, Zn, EDTA. Specific and selective targeting to pathogen-generated triglyceride lipase provide “biofeedback” based pathogen-activated mechanism of “suicide” and by utilizing an essential host-pathogen interaction, and therefore unlikely to develop tolerance and resistance.

According to some embodiments, the invention provides TriAza-based microbiome enriched topical formulations.

According to some embodiments, in psoriasis, TriAza balances protective microbiome via eradicating virulent Staph. Aureus (SA) and reducing SA-induced inflammation.

According to some embodiments, anti-aging TriAza-based treatment is synergistic with Nitrosomonas eutropha, while proven by skin appearance, skin barrier and wrinkle improvement.

According to some embodiments, in photo-aging, TriAza combined with plant extract fermented Lactobacillus buchneri reduces UV-B induced cytokine and ROS release, thus stabilizing melanin.

According to some embodiments, TriAza-based formulation further comprises one or more active substances including, without limitation, microbiome active agent (probiotic, postbiotic, prebiotic), anti-infective agents, topically/dermatologically active agent, antifungal agents, antiviral agents, antibiotics, adaptogens, growth factors, cytokines, chemokines, nucleic acids, vitamins, minerals, anti-inflammatory agents, anesthetics, moisturizers, extracellular matrix proteins, enzymes, stem cells from plants, extracts from eggs and eggshells, honey (preferably date honey), hyaluronic acid, botanical and medicinal mushroom extracts (i.e. polysaccharides, sterols, phenols, organic acids, polyphenols, stilbenes, flavonoids, sterols, waxes, squalens, and other lipids and oils, triterpenes and glycosids/saponines, alkaloids, glucans, long chain, allicin, medium and short chain fatty acids, purines and derivatives of thereof, polyamines (e.g. spermine and spermidine), and antiproliferative agents, allicin, dicarboxylic acids, and skin penetration enhancers.

According to some embodiments, an active agent is in the range of about 0.1 wt % to about 40 wt % of the total composition. According to some embodiments, an active agent is in the range of about 0.5 wt % to about 40 wt %; 2 wt % to about 40 wt %; 5 wt % to about 40 wt %; 10 wt % to about 40 wt %; 15 wt % to about 40 wt %; 20 wt % to about 40 wt %; 30 wt % to about 40 wt %; 0.5 wt % to about 35 wt %; 0.5 wt % to about 30 wt %; 0.5 wt % to about 25 wt %; 0.5 wt % to about 20 wt %; 0.5 wt % to about 15 wt %; and, 0.5 wt % to about 10 wt %.

According to some embodiments, disclosed are TriAza-based formulations further comprising retinoids (e.g. vitamin A, acitretin, isotretinion, tretinion and tazarotene); peroxides (e.g. benzoyl peroxide); antibiotics (e.g. tetracycline, clindamycin, erythromycin, metronidazole, sulfacetamide, doxycycline, oxytetracycline, minocycline, and trimethoprim); hormones (e.g. co-cyprindiol); adapalene; nicotinamide; salicylic acid; phenylephrine hydrochloride, pramoxine HCL, lidocaine, a corticosteroid, vitamin D and derivatives thereof; antralin, and calcineurin inhibitors, elastin, hyaluronic acid, collagen or chitosan, steroids or hormones or pheromones (e.g. co-cyprindiol, estradiol, testosterone, or derivatives of thereof); an anesthetic agent, a hemostatic agent, an anti-inflammatory agent, a hemp-based extract (cannabinoid), tryptophan/indole/melatonine derivative, serotonin, dopamine derivative, a deodorant, a pain relief agent, a UV protection agent, Zinc oxide, and combinations thereof, aloe vera, and combinations thereof.

According to some embodiments, topically active agent may be selected, without limitation, from antibiotics, antibacterial agents, antivirals, anti-parasitic agents, antifungals, anesthetics, analgesics, painkillers, anti-allergic agents, anti-acne, antimitotic, antipruritic drugs, antihistamines, immunosuppressants, corticosteroids, keratolytic, anti-angiogenics, anti-inflammatory drugs, phosphodiesterase 4 inhibitors, anti-cancer drugs, anti-neoplastic drugs, anthracene derivatives, psoralens, anti-proliferative drugs, vitamin D analogues, anti-alopecic (prostaglandin analogues), anti-heretic's, photosensitizers, de-pigmentants, hormones, vasoconstrictors, and a mixture or two more thereof.

According to some embodiments, disclosed are TriAza-based compositions further comprising acetaminophen, acetylsalicylic acyclovir, adapalene, alclomefasone, alpha-acid, acitretin, tocopherol, amcinonide, amorolfine, amphotericin B, tetracycline, benzoyl peroxide, betamethasone, brimonidine, calcipotriol, calcitriol, ciclopirox, clindamycin, crisaborole, clobetasol, crotamiton, cyproheptadine, dapsone, desonide, diclofenac, diflucortolone, difluprednafe, dioxyanthranol, econazole, efinaconazole, erythromycin, estradiol, etretinate, fluocinolone acetonide, fluticasone, fusidic acid, momefasone, glycolic acid, glycyrrhefinic acid, halobefasol, hydrocortisone, hydroquinone, ibuprofen, imiquimod, isotretinoin, ivermectin, kefoconazole, kojic acid, lactic acid, lidocaine, malic acid, mequinol, mefhoxsalene, metronidazole, miconazole, minoxidil, ocfopirox, oxymefazoline, pilocaine, pyridoxine, progesterone, retinol, pimecrolimus, resiquimod, rucinol, tacrolimus, tazarofene, terbinafine, tetracaine, thenaldine, fravopost, tretinoin, trimeprazine, trimeprazine, trifarofene, zinc pyrithione, and salts or derivatives of these active ingredients, and a mixture of two or more thereof.

According to some embodiments, the anti-infection agent may be, without limitation, antibiotics (e.g. tetracycline, clindamycin, erythromycin, metronidazole, sulfacetamide, doxycycline, oxytetracycline, minocycline, and trimethoprim); or antibacterial agent: alcohols, chlorine, peroxides, aldehydes, triclosan, triclocarban, benzalkonium chloride, linezolid, quinupristin-dalfopristin, daptomycin, oritavancin and dalbavancin, quinolones, and moxifloxacin, essential oil or terpene; or anti-fungal agent: the antifungal composition is selected from the group consisting of: clotrimazole, econazole, miconazole, terbinafine, fluconazole, ketoconazole, mophotericin, nystatin, sporanox, difulcan, terazol, intraconazole, mycostatin, boric acid, tioconazole, undecylenic acid, tolnaftate, imidazole, luliconazole, tavaborole, allylamine, amorolfine, oxiconazole, gluconazole, ciclotirox, naftifine, amphotericin B, sulconazole, butenafine, sertaconazole, efinacanazole, a derivative or prodrug thereof, and combinations thereof; chitosan, sulfadiazine, silver sulfadiazine, silver nitrate, silver nanoparticles, and combinations thereof.

According to some embodiments, the above compositions further comprise antiaging cosmetic ingredients such as, without limitation, alginate oligosaccharides, hydrogenated algin, Glutrapeptide, Indole, melanin mimetics, artemisia extracts, Pinolumin, vanilloid-1 receptor agonists, neurotransmitters agonists, bioactive ingredients (e.g. D-tyrosine, hyaluronic acid derivatives; perlecan and agrin derivatives); natural botanical extracts (e.g. polyphenol stilbenes); grifolin derivatives; thioredoxin and other bioactive proteins, naturally derived or synthetic approved peptides and analogous of thereof (e.g. collagen tripeptide, Lipotec SNAP-8TM Peptide; leucylproline, Lipotec ARGIRELINE® Peptide; Lipotec Inyline Peptide; Bachem Cosmetic Peptides, SPACE-peptide carriers, Prospector BONT-L Peptide Solution (PF), argireline®, acetyl hexapeptide-3, BODYFENSINE® Peptide., BASF Skinasensyl® LS 9749, dipeptides (e.g. derivatives and analogues of kyotorphin; lipid dipeptides, tetrapepetides); branded cosmetic proven active ingredients, i.e. BASF LS Skinasensyl; SensAmone P5; Neurobiox™ The Skin Biosurfacer, CRODA Calmosensine™; RAHN DEFENSIL®-SOFT, Sederma Prospector Calmosensine™ SP, Infinitec X50® Myocept Evercool® Skin; Codif STOECHIO.

According to some embodiments, disclosed TriAza as an emollient and/or skin barrier protectant.

According to some embodiments, TriAza is sebum mimetic functional emollient characterized by amphiphilic, pleasant scent and smooth texture pleasant when applied to the skin.

According to some embodiments, TriAza protects skin by forming a thin hydrophobic film on the surface of the skin to retard transepidermal loss of moisture.

According to some embodiments, as emollient, TriAza provides an occlusive barrier for AD skin, retains moisture, and protects from irritants.

According to some embodiments, as emollient, TriAza emollient functionality is additive and optionally synergistic to its antimicrobial, anti-itch and anti-inflammatory actions.

According to some embodiments, additional emollient ingredients may be added to TriAza formulation if designed as a functional sebum mimetic, and/or skin barrier protectant. The non-limiting examples include lanolin, mineral oils, olive oil, petrolatum ceramide, paraffin and silicone, collagen, elastin, glyceryl stearate and shea butter.

According to some embodiments, humectants may be added to attract water vapor to moisturize the skin such as glycerin, alpha hydroxyl acids and sorbitol.

According to some embodiments, osmo-protectants can be added, including, without limitation, glycosides, Phyto-protectants, trehalose and ectoin, an organic osmolyte.

According to some embodiments, due to the environmentally safe method of sourcing and production, TriAza is safe and an appropriate emollient for atopic dermatitis and eczema, and KP patients to improve acceptability and adherence for emollient treatment.

According to some embodiments, TriAza fortifies olive oil-process of oxidation.

According to some embodiments, TriAza based formulations further comprise Histone deacetylase inhibitors (HDACi), such as phenylbutyrate, valproic acid (VPA) or vorinostat (SAHA), which exert several immunostimulatory properties, and contribute at least in part to their anticancer effect.

According to some embodiments, TriAza based formulations further comprise alpha hydroxy acids (AHAs), poly-AHAs, complex poly-AHAs, retinoids, polysaccharides, anti-enzymatic agents, antioxidants (including ascorbic acid, pycnogenol, ursolic acid, vegetable isoflavones, vitamin E, coenzyme Q10, lipoic acid, resveratrol, 1-carnosine and taurine), choline, caffeine, acylcarnitine, as well as various plant and medicinal mushroom extracts.

According to some embodiments, the invention provides a composition designed to deliver at least one biomolecule and/or chemical entity/and/or biological sample to a subject in need of such delivery, comprising an amount of a carrier having Formula 1

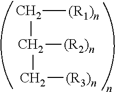

Wherein each of R₁, R₂, and R₃ is independently selected from the group consisting of discarboxylic acid, azelaic acid; and wherein n is n=1 (one or more) and wherein the amount of said carrier is effective to deliver said biomolecule and/or biological sample to a predetermined target.

According to some embodiments of the above composition, the composition may be, without limitation, an anti-septic, composition, a chemical composition, a pharmaceutical composition, a cosmeceutical composition, an edible composition, and a composition comprising microbiome. In the context of the invention, the term “microbiome” refers, without limitation, to the microorganisms in a particular environment (including the body or a part of the body). Microbiome also refers to the combined genetic material of the microorganisms in a particular environment. Microbiome is considered as a term that describes the genome of all the microorganisms, symbiotic and pathogenic, living in and on all vertebrates. A non-limiting example of microbiome includes Lactobacillus, Bacillus, and Bifidum, and balance of host microbiota or/and transplanted administered probiotic compositions that do not overexpress lipase as a major virulence factor to induce biofilms and opportunistic and invading, contamination induced pathogens.

According to some embodiments, the present invention further provides a lipase-hydrolyzable (lipase-mediated) site-targeting sytem releasing upon hydrolysis an inert, or non-inert, carrier compound (referred to as “carrier component”) and an active compound (referred to as “active ingredient”), selected from the group consisting of:

According to some embodiments, a lipase-hydrolyzable triglyceride ester comprising an inert carrier component (glycerol moiety) and an active component (fatty acid moiety). For example, a component, such as azelaic and/or retinoic acid is chemically attached to the glycerol backbone to form tri-retinoic, tri-azelaic and mixed di-retinoic, mono-azelaic, or di-azelaic, mono-retinoic ester derivatives.

According to some embodiments, the triglyceride ester of the present invention provides three molecules of active fatty acids per one mole of ester.

According to some embodiments, a lipase-hydrolyzable moiety comprising an antibiotic component (such as erythromycin, macrolide or lincosamide moiety) and an anti-acne active carrier (such as linoleic or azelaic acid moiety).

According to some embodiments, the ester of the present invention provides one molecule of anti-acne active fatty acid (serves also as a carrier component) and an antibiotic material per one mole of ester.

According to some embodiments, a compound comprising a moiety of an inert cholesterol or cholesterol-like carrier and a moiety of an antibiotic active ingredient (such as macrolide, preferably 14-membered macrolide, or lincosamide, preferably clindamycin).

Dosing is dependent on the severity of the symptoms and on the responsiveness of the subject to the active drug. The dose is determined by the attending physician, in consideration of age, sex, weight and state of the disease. According to some embodiments, the invention provides a process for production of acylglycerol of azelaic acid (“triazelaic glycerols”, triazelaines)—enriched mixtures (“TriAza”, or 3Azor, or TriAza, or 3Az) from an unsaturated fatty acid containing triglyceride starting material such as oleic oil.

According to some embodiments, the TriAza is produced using an ozonolysis or oxidation processes under alkine conditions. During the process, the ozonolysis of one mole of Triolein generated unsaturated ozonoids of Triolein, which are further oxidized and following breakage of unsaturated bonds-one mole triazelaine and three moles of pelargonic acids are obtained. In one embodiment, a pure TriAza may be produced by chemical liquid synthesis production.

According to some embodiments, the TriAza is produced according to processes described in examples 1 to 5, 10 and 11.

According to some embodiments, the obtained product TriAza is further modified by saponification, polymerization, emulsifying, suspension, gelation, and then is used as soaps, polymers, emulsions, suspensions gels in various cosmetic, natural-based pharmaceutical, nutraceutical and other practical compositions.

According to some embodiments, the invention provides a new a chemical conjugate, hydrolysable by a bacterial lipase, that upon hydrolysis by said lipase releases an active ingredient which is effective in treating a pilosebaceous unit associated disease, preferably the acne pathogen. Upon the hydrolysis, the compounds of the invention also release an inert, or non-inert, carrier component.

According to some embodiments, the invention provides a bacterial lipase-hydrolysable ester providing upon hydrolysis (1) an inert, or non-inert carrier component selected from the group consisting of glycerol, a mono- or dicarboxylic acid and cholesterol or cholesterol-like compound; and (2) an ingredient active in a treatment of a pilosebaceous unit disease derived from a group consisting of an antibiotic material or a fatty acid compound exhibits an anti-acne activity. Same anti-acne fatty acid may be aliphatic, cyclic, aromatic, saturated, unsaturated, mono, di and polycarboxylic acids and a mixture thereof.

According to some embodiments, the invention provides a prodrug conjugate hydrolysable by a lipase of P. acne or any other lipase produced by lipase-producing microorganisms specific to a pilosebaceous unit. A non-limiting list of lipase-hydrolysable conjugates includes: a triglyceride ester releasing three molecules of anti-acne active fatty acids; an ester releasing one molecule of anti-acne active fatty acid and an antibiotic material; and a cholesterol ester releasing an antibiotic material. Each of said conjugates releases upon exposure to a lipase specific to a pilosebaceous unit an ingredient active in the treatment of a pilosebaceous unit disease, including, without limitation acne, baldness, seborrhea, and hirsutism. In one embodiment, the triglyceride conjugate which releases azelaic acid is useful in the treatment of hormonal-dependent acne. In one embodiment, a non-glycerol conjugate is useful in the treatment of infectious acne.

According to some embodiments, the invention provides a cosmetic preparation based on special acylglycerols, preferably triazelaine, that have improved bio-protective properties. The bio-protective properties of the composition are based, without limitation, on dual mechanism of action, namely, release of aseptic azelaic acid and substrate competition of triazelaine with natural triglyceride substrates.

According to some embodiments, the obtained triazelaines can be further incorporated in emulsions and can provide the preparations according to the invention with improved bactericidal and bacteriostatic properties against triglyceride lipase-generating microorganisms.

According to some embodiments, the present invention provides novel compositions and their synthesis for topical treatment of disorders of pilosebaceous unit.

According to some embodiments, the invention provides a nutrition or topical emulsion comprising the structured triglyceride of the invention. The emulsion composition according to the invention can comprise a natural biologically compatible emulsifier, such as, without limitation, lecithin, phosphatidylcholine, phosphatidyl ethanolamine or mixtures thereof. In one embodiment, the emulsion further comprises vitamin E, preferably, alpha tocopherol. In one embodiment, the emulsion further comprises a pharmaceutically acceptable non-natural surfactant. A non-limiting list of suitable surfactants includes TYLOXAPOL; POLOXAMER; POLYOXYL 40 Stearate; POLYSORBATE, TWEEN, PLURONIC F-68, polyoxyethylated oils, and, poloxamines. In one embodiment, the oily mixture comprises the structured triglycerides, and also an anti-oxidant. Typically, the emulsions of the present invention are filtered through a membrane filter or sterilized by heating up under inert gas atmosphere such as, for example, nitrogen.

According to some embodiments, the composition of the invention is cosmetically or dermatologically acceptable, namely non-toxic and capable of being applied to human skin, including, without limitation, the inside of the eyelids, vaginal area, or the lips.

According to some embodiments, the compositions further comprise at least one emulsifier. The addition of emulsifiers improves the incorporation of the triazelaine into the final composition. In one embodiment, the emulsifying agent improves lipase-mediated release of azelaic acid from triazelaine.

According to some embodiments, the active agent may be used in a formulation/delivery system that may be applied to human skin or coated on the textile or disposable sheet surface for the eradication of skin microflora, including corynebacterial, Staphylococcus, proioniumbacteria, Candida, reduction or elimination of body irritation, inflammation, infection, malodor, itching, chafing or moisture. By application of disclosed herein antimicrobial topical formulation comprising of acylglycerols of azelaic acids, and/or other antimicrobial “triglyceride-mimicking “substances”, named herein TriAza substances, deodorant and anti-irritating activity may be reached, and thereby may be used to mitigate odor, or topical or skin irritation in a subject.

According to some embodiments, the TriAza containing formulation can be used as coating for disposable absorbents such as, without limitation, diapers, textile, medical devices and other relevant surfaces.

According to some embodiments, the active agent of the invention may be used in an amount effective to inactivate microflora. Only those microflorae capable of catabolizing triglycerides, especially Triolein s by specific triglycerides lipase. According to some embodiments, the active agent may be present in an amount ranging from 0.01 to 20% by weight of the composition. According to some embodiments, the active agent may be present in an amount ranging from 0.1 to 90%.

According to some embodiments, the invention provides process for the preparation of the composition comprising triazelaine, the process comprising oxidizing Triolein in a vegetable oil without hydrolyzing the acyl glycerol bond.

According to some embodiments of the above process, a non-limiting list of vegetable oils is includes corn oil, cotton seed oil, olive oil, almond oil, jojoba oil, argan oil, palm kernel oil, rape seed oil, safflower seed oil, evening primrose oil, soybean oil, sunflower seed oil, avocado oil, sesame seed oil, coconut oil, arachis oil, and castor oil.

According to some embodiments, the active agent of the invention TriAza, exhibit multifunctionality which is based on its nature as a (a) lipid-based emollient (i.e. skin and mucosal barrier protection) and as a (b) microbiome balancing, by maintaining low risk of overgrowth of lipase producing microbiome strains.

According to some embodiments, the active agent of the invention TriAza is targeted to treat inflammation/irritation symptoms caused by at least one of the following pathological processes (1) skin barrier destroyed and/or (2) lipase overexpressing microbiome overgrowth.

According to some embodiments, the composition comprising TriAza, or C9 derivatives of thereof, provides a safe and effective “anti-irritation”, or “anti-inflammatory treatment.” Said “anti-irritation” treatment meaning: (i) reducing the severity of the irritaton, (ii) ameliorating a symptom of the irritation, and/or (iii) shortening the duration of the irritation, (iv) decreasing recovery time from a skin irritation.

According to some embodiments, the said anti-inflammatory treatment includes relief, or temporary relief of pain, redness, or discomfort due to mild to moderate skin inflammation.

According to some embodiments, the use of TriAza composition for the treatment for sunburn induced inflammation, or other kind of inflammatory symptoms, such as irritation, burning sense, itching, redness, by administering to the patient in need a combined therapeutically effective amount of TriAza and optionally OTC (over-the-counter) local analgesic (e.g. lidocaine), or/and NSAID (e.g. piroxicam), steroid (e.g. cortisone), or/an antihistamine.

According to some embodiments, the use of TriAza composition for the reduction of symptoms of microbial overgrowth induced irritation, for example Candida induced anti-irritation treatment of topical skin or mucosal irritation caused by Candida or other microbial pathogen, or mixed biofilm caused overgrowth or infection-induced by lipase-overexpressing unbalanced commensal microbiome, such as such as, without limitation, Candida, Pseudomonas, and Staph. Infection can be nosocomial infection caused by resistant strains (e.g. MRSA).

According to some embodiments, the active agent of the invention TriAza can be given as a single composition or as a combination with other anti-infective microbicidal or/and antibiotic treatments, such as, without limitations clotrimazole, mupirocin, streptomycin, iodine, permanganate.

According to some embodiments, the use of TriAza composition is for the treatment of a vaginal yeast infection (candidiasis) that causes irritation, discharge and intense itchiness of the tissues at the vaginal opening. Preferable use of Triaza combination for vaginal candidiasis is combined with live or freeze-dried probiotic, or/and postbiotic, or/and antifungal such as, without limitations clotrimazole agents.

According to some embodiments, the use of TriAza composition is for the prevention and treatment of vaginal inflammation symptoms caused by postmenopausal dryness, a skin barrier estrogen reduction associated impairment, in combination with lactic acid or/and estrogen agents in single formulation or as a parallel regimen.

According to some embodiments, the use of TriAza composition is for prevention, reduction, shortening and elimination of symptoms of mucositis. According to some embodiments, the inflammation/irritation symptoms caused by mixed infection includes chemotherapy or radiation induced mucositis cause sores in the mouth, throat, stomach, rectum, or vagina due to inflammation of the mucous membranes. According to some embodiments, the symptoms are accelerated by degraded mucosal barrier due to cancer therapy and unbalanced overgrowth of lipase-overexpressing microbiome. According to some embodiments, TriAza composition is used in combination with local anesthetic, such as, without limitation lidocaine, and/or vasoconstrictor, such as, without limitation pseudoephedrine, and/or antihistamine, such as, without limitation loratadine. According to some embodiments, another symptom of cancer therapy induced inflammation, is skin dryness and inflammation symptoms.

According to some embodiments, preferable anti-mucositis regimen for the prevention of mucositis and skin barrier inflammation and overgrowth symptoms is administered (i) prior, (ii) during and (iii) post-cancer treatment.

According to some embodiments, the use of TriAza compositions for the prevention can be formulated in frozen, ice cream-like formulations to be taken immediately before or during chemotherapy, radiotherapy to protect mucosal and barrier:

According to embodiments, some TriAza compositions are administered to patient before, during and after cancer therapy sessions for best results. According to some embodiments, TriAza compositions should be administered prior to cancer treatment-preferably preventive eradication of lipase-expressing microbiome by application of TriAza combination, as a stand alone or in combination with other antimicrobials.

According to some embodiments, TriAza compositions are administered as cooled formulations during chemotherapy especially during radiotherapy sessions, preferably with local anesthetic and/or vasoconstrictor as above.

According to some embodiments, TriAza compositions are administered during post-treatment, and includes pro-; pre-; post-biotic for microbiome balance.

According to some embodiments, the invention provides screening of C9-MOCT-tripelargoinin (TG9—can be defined as “derivative” of C9 MOCT-TriAza) and C7-MOCT-triheptanoin (TG7—can be defined as a “precursor” of TriAza) that also served as substrates for hydrolysis microbial (Candida, Pseudomonas) lipase and inhibited culture growth in comparison to favorable substrate Triolein that promotes growth. TG9 lipase hydrolysis end product-MOCFA-monocarboxylic nonanoic acid-Pelargonic acid exhibited MIC similar to MIC of MOCFA-dicarboxylic nonanoic acid-Azelaic acid, TriAza MIC was the highest and TriAZa was the only compound between MOCT and MOCFA that exhibited not only inhibition of growth measured as MIC, but also stopped growth of lipase overexpressing pathogens, as measured by MBC.

According to some embodiments, the invention provides selection of oil with low comedogenic index 1-2, not more than 3 and validated carrier oil fatty acid composition for providing (i) sufficient level of skin barrier protective lipids for delivery, uptake and replacement of naturally reduced cell barrier triglycerides (especially in mature skin in females over age 50) and other skin barrier lipid mimetics; (2) sufficient level of protective lipids, e.g. triolein as a precursor of oleic acid that serves novel “fasting mimetic”; (3) low level of peroxides and sufficient level of antioxidants in the carrier oil, raw material carrier oil should be sufficiently analytically validated and preferably comprised at least two sources of oil with complementary sensorial and functional properties.

According to some embodiments, this invention provides triolein (a major preferable ingredient of carrier oil, e.g. olive oil, sunflower oil, sesame oil), as a sufficient nutrient for survival of lipase producing commensal microbiota under physiological balance-once lipase is not under virulent overexpression-is a part of skin microbiome diversity-related to healthy skin without “aging appearance” symptoms.

According to some embodiments, the multifunctionality of TriAza/and C9 MCT/OCFA composition is based on: (1) regulation of cellular barrier and anti-inflammatory activity; (2) preserving cellular energetic status based on anaplerotic nature of odd chain fatty acids as substrate to Krebs cycle regeneration and as a medium chain fatty acid source for ketogenic energy; (3) microbiome balance through preventing overgrowth of lipase overexpressing commensal microbiome (Staph spp., Candida) and pathogenic species (Aspergillus; MRSA; Pseudomonas). Therefore, such multifunctionality targets three major skin ageing causes: (1) skin barrier loss translated in aging appearance related symptoms-skin dryness, pigmentation, wrinkles and loss of elasticity; mitochondrial dysfunction and microbiome disbalance that also impact “aged appearance” of mature skin.

According to some embodiments, the disclosed compositions may be used to treat various dermatological disorders such as, without limitation hyperkeratosis, photo-aging, bums, donor site wounds from skin transplants, ulcers (cutaneous, decubitus, venous stasis, and diabetic), psoriasis, skin rashes, and sunburn photoreactive processes. According to some embodiments, the topical therapeutic compositions of this invention may also be used orally in the form of a mouth wash or spray to protect and accelerate the healing of injured oral tissue such as, without limitation mouth sores and bums, treatment of various such as, dermatological disorders without limitation hyperkeratosis, photo-aging, and sunburn photoreactive processes.

According to some embodiments, the mechanism of said treatment in the disclosed invention is directed to a method for preventing and reducing injury to mammalian cells, and increasing the resuscitation rate of injured mammalian cells, which comprises the steps of providing a therapeutic wound TriAza healing composition which comprises (a) carrier preferably vegetable oil enriched with Triolein (at least 20%) and comedogenic index less than 2, (b) MOCT/OCFA, wherein MOCT is TriAza and/or TG9-tripelargonin, or/and TG7-triheptanoin, and OCFA-nonanoic acids, salts and esters or diesters of thereof, branced and/or linear, and (c) a mixture of anti-ageing active agents, e.g. antioxidants or/and enhancers targeting protection of cellular barrier (e.g. carrier oils) or maintainers of mitochondrial energy balance (coQ10, acyl-carnitine) or derivatives of thereof assiting the for resuscitation of injured mammalian cells, and contacting the therapeutic wound healing composition with the mammalian cells.

According to some embodiments, injury of mammalian cells which causes epidermal damage such as, without limitation lesions, incisions, wounds in which the skin is broken by a cutting instrument and lacerations, wounds in which the skin barrier (epidermal cells barrier) is broken by lesion induced by endogenous degenerative process (e.g. diabetic ulcers, immune-related psoriasis, or allergic induced pruritis, aging induced dryness and sores, inflammation); or by external, i.e. mechanical tension (pressure wounds, shaving wounds, surgery, etc.), burns, or by photo UV radiation-gamma radiation-induced damage; or by xenobiotic infection.

### EXAMPLES

### Example 1: Synthesis of Azelaic Acid Glyceryl Triester

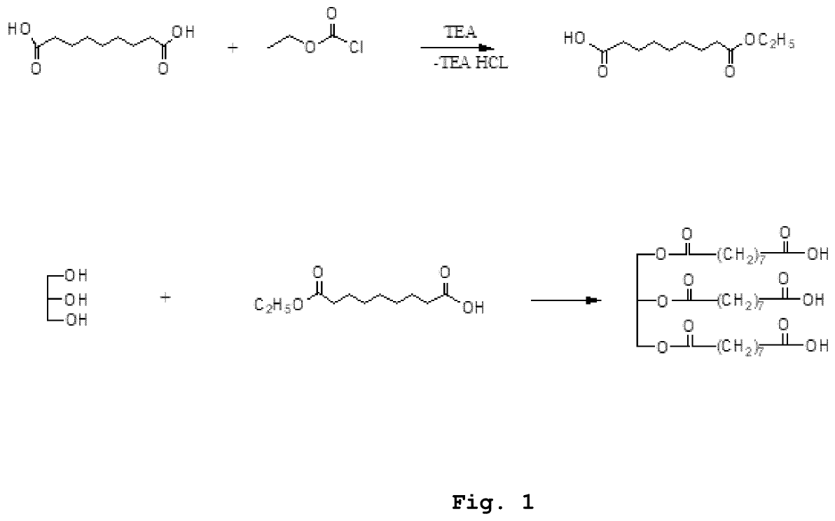

Ethyl chloroformate (about 10 mmol) was added at −5-0° C. to a solution of azelaic acid (about 10 mmol) and triethylamine (TEA, about 10 mmol) in dry tetrahydrofuran (THF, about 40 ml). The mixture was separated by centrifugation and the supernatant was added to a solution of dry glycerol (about 2.5 mM). The resulting mixture was stirred for about 1 hour at 25° C. and for 1 hour at 50° C. Then water (about 30 ml) was added to the cooled reaction mixture, followed by evaporation under reduced pressure. The product was extracted with chloroform (2× about 30 ml). The resulting organic solution was dried with sodium sulphate, followed by evaporation, which resulted in a fine white crystalline material. The yield was 10%. FIG. 1 shows the schematic pathway for synthesis of azelaic acid glyceryl triester.

### Example 2: Synthesis of Azelaic Acid Glyceryl Triester

Alternative method of synthesis of azelaic acid glyceryl triester

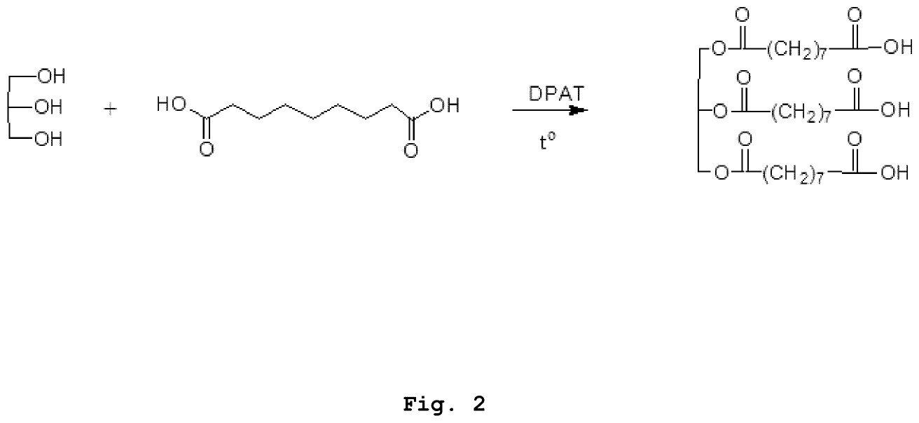

A solution of 2.06 g (10 mmol) of dicyclohexylcarbodiimide (DCC) in 15 ml of dry MeCl₂ was added very slowly (dropwise) to the solution of 0.23 g (2.5 mmol) of dry glycerol, 0.03 g (0.25 mmol) N, N-dimethylaminopyridine, and 1.65 g (8.8 mmol) of azelaic acid in dry CH₂Cl₂ (30 ml) and 5 ml of dry tetrahydrofuran (THE). The resulting suspension was stirred overnight at 25° C. Dicyclohexyl urea (DCU) was filtered in vacuum and the solvent was evaporated under reduced pressure without heating. The residue was re-dissolved in THF and non-dissolved material (DCU) was separated. THF was evaporated to dryness until an oily residue was left in the flask. This oily residue solidified after lyophilizing. The resulting material purified on preparative thin layer chromatography using chlorophorm/methanol/water (65:25:4, v/v/v) as the developing solvent. Yield: around 25-30%. NMR analysis of the resulting compound demonstrated relevant peaks. FIG. 2 shows the schematic pathway for this synthesis of azelaic acid glyceryl triester.

### Example 3: Synthesis of Retinoic Acid Glyceryl Triester

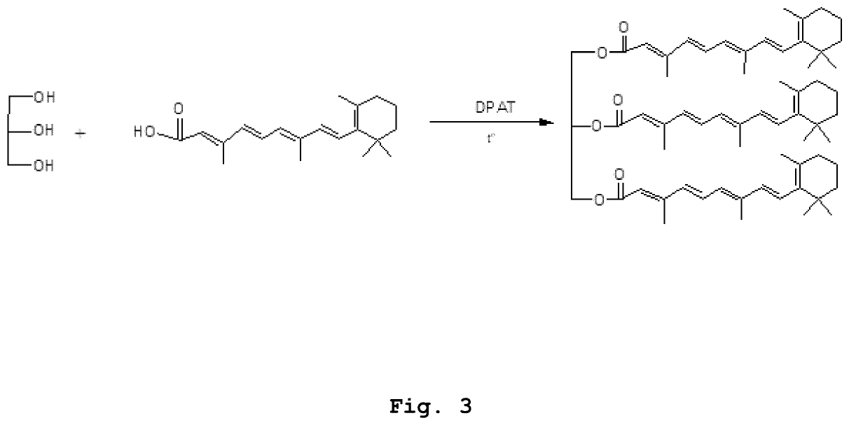

A solution of dicyclohexylcarbodiimide (DCC, about 20 mmol) in dry dichloromethane (about 30 ml) was added dropwise to a mixture of dry glycerol (about 5 mmol), N, N-Dimethylaminopyridine (DMAP, about 0.5 mmol) and retinoic acid (about 17.5 mmol). The resulting suspension was stirred overnight at 25° C. The resulting solution was filtered and methylene chloride was evaporated under reduced pressure without heating. The product was recrystallized. FIG. 3 shows the schematic pathway for this synthesis of retinoic acid glyceryl triester.

### Example 4: Synthesis of 1-Azelaic Acid 2,3-Retinoic Acid Glyceryl Triester

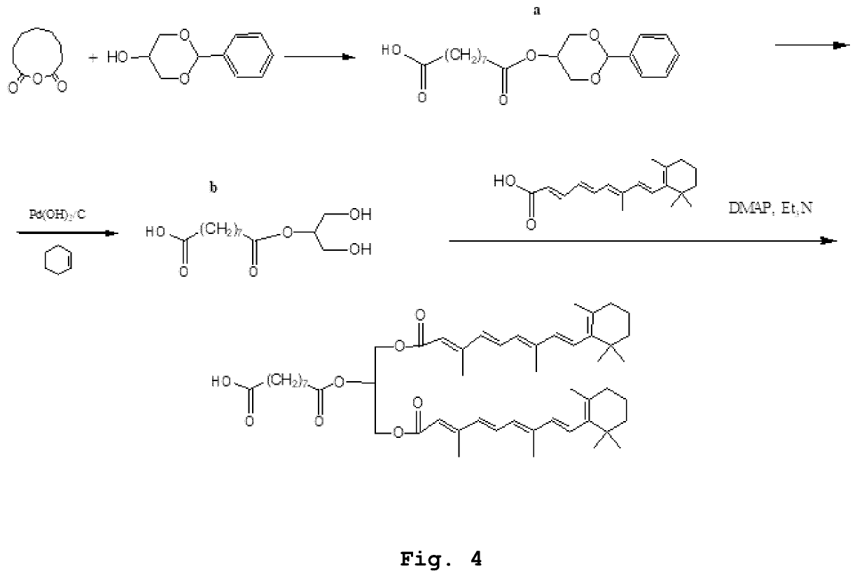

A solution of dicyclohexylcarbodiimide (DCC, about 7 mmol) in dry dichloromethane (about 20 ml) was added dropwise to a mixture of 2,3-isopropylideane-sn-glycerol (about 5 mmol), N, N-Dimethylaminopyridine (DMAP, about 1 mmol) and azelaic acid (about 6 mmol) in dichloromethane (about 30 ml). The suspension was stirred for about 12 hours at 25° C. The resulting precipitate was filtered, washed with water and lyophilized. The dry product was dissolved in dry dichloromethane (about 30 ml) and slowly added to the mixture. The suspension was stirred overnight at 25° C. After filtration of the precipitate, dichloromethane was evaporated under reduced pressure and the crude product lyophilized and subsequently recrystallized. FIG. 4 shows the schematic pathway for this synthesis of 1-azelaic acid, 2-3-retinoic acid glyceryl triester.

### Example 5: Synthesis of 2′-O-Erythromycin Ester of Linoleic Acid 2′-O-Linoleioyl Erythromycin

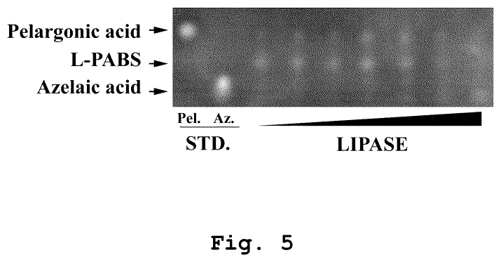

To an ice cooled solution of 1.0 mmol of methyl ester of erythromycin in 50 ml of CHCl₃, 1.1 mmol of Et₃N was added dropwise in argon atmosphere. After 3 min, a solution of 1.1 mmol of the linoleic acid in 35 ml of CHCl₃ was added at 0° C. and then 1.1 mmol of dicyclohexylcabodiimide and 1.1 mmol of hydroxybenztriazole were added as solids. Finally, 35 ml of N, N-dimethylfomamide (DMF) was added and the mixture was stirred for 2 h at 0° C. and for further 40 h at room temperature. Ethyl acetate (50 ml) was added, the mixture was filtered, concentrated in vacuo, and was filtered again. Ether (30 ml) was added and the mixture was extracted with water, 1% KOH solution and water. The organic phase was dried by MgSO₄, evaporated and the residue is chromatographed on a silica column (h=30 cm, d=3.2 cm). FIG. 5 shows the schematic pathway for 2′-O-Erythromycin ester of Linoleic Acid.

### Example 6: Lipase-Mediated Activity

To determine the affinity of a bacterial lipase to the active ingredient according to the embodiments of the invention, the ability of the lipase to utilize TriAza as a substrate was tested. For this purpose, commercially available lipase from P. cepacian was used. Increasing amounts of the enzyme were mixed with a fixed concentration of the substrate, and incubated for a few hours at 37° C. The enzymatic activity was monitored by TLC, by registering the production of azelaic acid. It was found that increasing concentrations of the enzyme are coupled to the gradual accumulation of azelaic acid at the expense of TriAza (FIG. 5). The results suggest that TriAza could be utilized as a substrate for bacterial lipase activity.

### Example 7: Lipase-Mediated Specificity

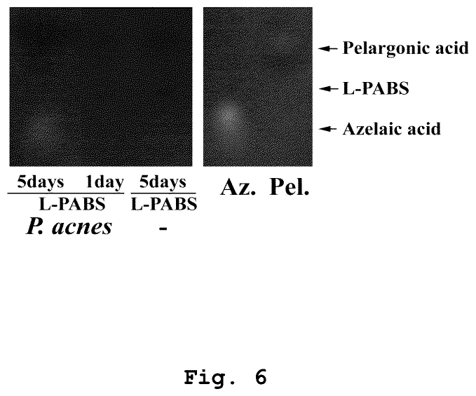

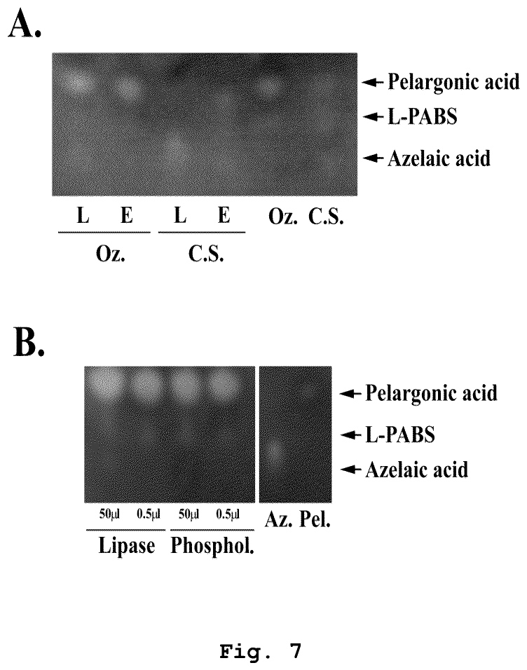

Pure pro-drug activity of designed molecules implies their high specificity towards the target enzyme. TriAza represents the glycerol ester of a specific fatty acid that might be recognized by a mammalian esterase or other triglyceride-utilizing lipase. To establish specificity of bacterial lipase to TriAza, the lipase activity from P. cepacia to that of mammalian esterase and phospholipase A2 was compared. The enzymes, in equally active amounts, were exposed to the fixed concentrations of TriAza. The extents of enzyme activities were registered according to the level of generation of azelaic acid. It was found that in contrast to the lipase, neither esterase nor phospholipase A2 could produce azelaic acid (FIGS. 6-7). These results indicate that consumption of TriAza by bacterial lipase is highly specific.

### Example 8: Antibacterial Activity

TriAza was designed as a pro-drug of azelaic acid, a known antibacterial agent, for the treatment of skin disorders associated with pathogenic propagation of bacteria. For example, the development of Acne vulgaris is tightly associated with extensive accumulation of P. acnes and Corynebacterium acnes-C. acnes in the sebaceous gland, a milieu of the disease. To determine the antibacterial activity of TriAza against P. acnes, the minimal bactericidal concentration (MBC) of TriAza was determined. The effect of TriAza was compared to the MBC of azelaic acid and benzoyl peroxide (BPO), two main competitors used in the treatment of acne. We found that TriAza, as well as the reference molecules, showed an anti-microbial activity against P. acnes ranging between 0.5-5 mg/ml (Table I). Even though all the molecules behaved similarly, the activity of TriAza was found to be 2-fold stronger than that of azelaic acid, supporting the use of TriAza in the treatment of acne vulgaris.

TABLE 1
Activity of antibacterial agents against P. acnes

| Name | MIC* |
|---|---|
| Azelaic acid | 3.75 mg/ml |
| BPO | 0.5 mg/ml |
| TriAza | 1.95 mg/ml |

*MIC designates a minimal inhibitory concentration for tested bacteria. MIC of L-PABS is equal to its minimal bactericidal concentration (MBC). MIC of L-PABS that is demonstrated in the table characterizes an antibacterial activity of batch #5.

To compare the antimicrobial activities of various batches of TriAza we determined MIC of number of batches that were prepared with non-principle modifications. Selected batches were labeled as TriAza-3, TriAza-6, TriAza-7 and TriAza-19 and supplied to the growth medium of P. acnes. A minimal inhibitory concentration of the batches was monitored and compared to each other. We found that all samples possessed similar antibacterial activity with small deviations (Table II). For further analysis, batch TriAza-19 was chosen as a batch containing minimal amount of pelargonic acid.

Since TriAza is designed for marketing as a soap, decided to define an antibacterial activity of its soap. To this end, sodium salt (a soap) of the most active batch of TriAza (TriAza-19-Na) was prepared and MBC of the material was determined. We have found that the TriAza-19-Na exhibits both anti-bacterial and -bactericidal activities, yet less significant than that of TriAza-19 itself (Table 1). This finding indicates that soap of TriAza can be used in anti-P. acnes treatment, although with a less anti-bacterial activity. It should be stressed that despite of decreased activity of TriAza soap it is still higher than that of azelaic acid (Table 1 and 2).

TABLE 2
Activity of various TriAza batches and their soaps against P. acnes

| Name | MBC (mg/ml) |
|---|---|
| TriAza-3 | 1 |
| TriAza-3-Na | 4 |
| TriAza-6 | 2.8 |
| TriAza-6-Na | 4 |
| TriAza-7 | 2 |
| TriAza-7-Na | 6 |
| TriAza-19 | 1 |
| TriAza-19-Na | 4 |

### Example 8: Antibacterial Specificity: Range of Activity

Since TriAza possesses antibacterial activity, the extent of its specificity to P. acnes was determined. For this purpose, a few major skin pathogens were selected and tested for sensitivity to TriAza. We chose Staphylococcus aureus (S. aureus) and Candida albicans (C. albicans), representing bacterial and fungal genera, respectively. Both organisms express lipase. The bacteria and fungi were exposed to various concentrations of TriAza and its sodium salt, and both the MICs and MBCs of the agents were statistically determined. We found that both organisms were sensitive to the material, with S. aureus exhibiting higher sensitivity. The level of TriAza activity was similar to that demonstrated against P. acnes. The slight difference in MICs of TriAza against the tested organisms can stem from the difference in their lipase activity. Our findings revealed that TriAza could be used in the treatment of various infectious diseases associated with S. aureus and C. albicans infections.

TABLE 3
Activity of TriAza and its soap against S. aureus and C. albicans *

| | MIC | MBC | MIC | MBC |
|---|---|---|---|---|
| | (S. | (S. | (C. | (C. |
| Name | aureus) | aureus) | albicans) | albicans) |
| TriAza-19 | 5 | 10 | 20 | 24 |
| TriAza-19-Na | 31 | 63 | 31 | 37 |

*** The parameters of MIC and MBC are presented in mg/ml calculations.

### Example 9: Determination of Optimal Conditions to Carry Out the Conversion of Trioleate to Triazelate

Trioleate in Olive oil was analyzed using HPLC-CAD method under following chromatographic conditions:
Detector: CAD
Column: C18, 150×4.6 mm, 5 micron.
Oven Temperature: 40° C.
Flow rate: 1.5 mL/min
Run Time: 26 min
Eluent A: Acetonitrile
Eluent B: Chloroform
Gradient profile is represented in Table 4:

TABLE 4
Gradient profile for acetonitrile (Eluent
A) and chloroform (Eluent B)

| Time (min) | Eluent A | Eluent B |
|---|---|---|
| 0 | 90 | 10 |
| 10 | 50 | 50 |
| 20 | 50 | 50 |
| 20.1 | 90 | 10 |
| 26 | 10 | 90 |

Diluent: Acetonitrile:Chloroform (90:10)

Typical chromatogram of Olive oil is shown in FIG. 1. Highest peak is Trioleate.

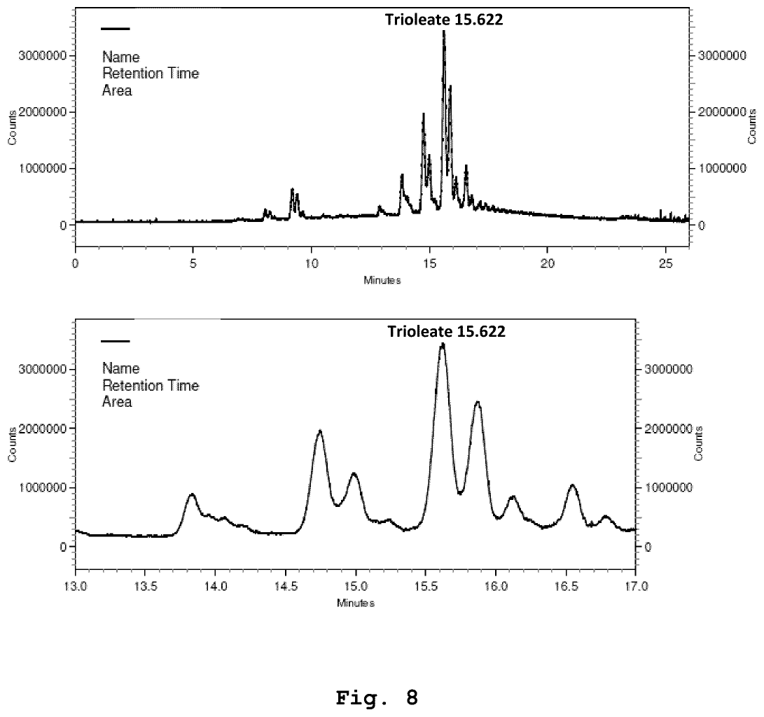

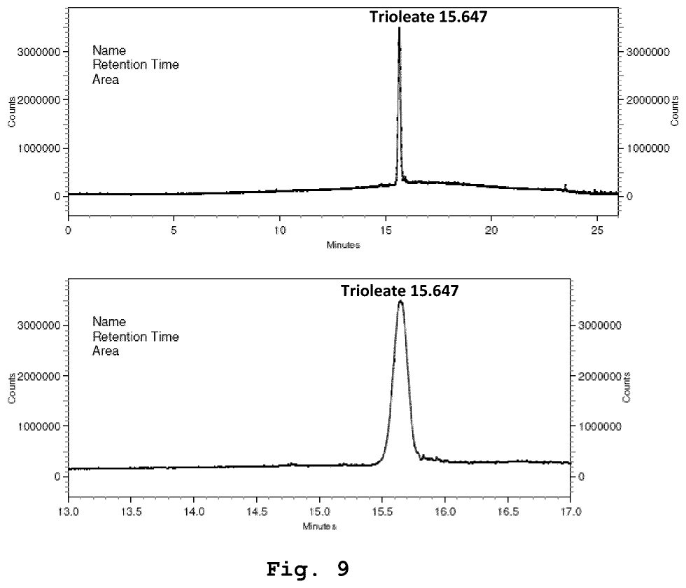

FIG. 8 shows typical chromatogram of Olive oil, while FIG. 9 demonstrates typical chromatogram of Trioleate.

Our analysis yielded 28% of Trioleate in Olive oil.

### Analysis of Pelargonic Acid

Pelargonic Acid was analyzed by two different methods: RP-HPLC and GC-FID. HPLC method has a serious limitation: oily phase cannot be analyzed directly while Pelargonic Acid has a low solubility in water.

### HPLC Conditions:

Detector: UV, 210 nm
Column: XBridge C18, 150×4.6 mm, 5 micron.
Mobile phase: Acetonitrile: Water: H₃PO4 (50:50:0.1)
Flow: 1.0 mL/min
Run time: 15 min
Column temperature: 35° C.

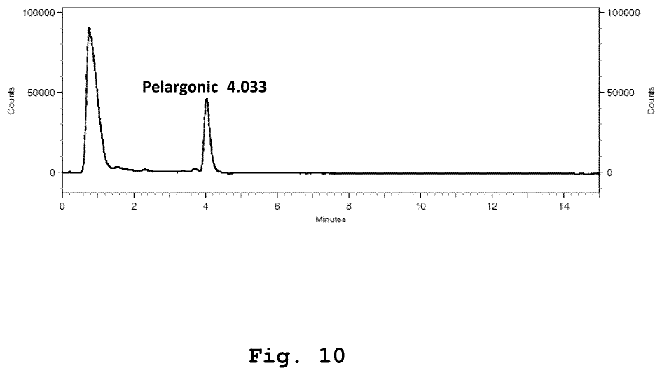

FIG. 10 demonstrates chromatogram of Pelargonic Acid in methanol by HPLC.

Pelargonic acid has much higher solubility in oils and organic solvents than in water therefore it is very difficult to extract that for analysis in HPLC.

### Gc Conditions:

Detector: FID
Column: Supelco, Nukol, 15 m×0.5 mm, 0.53 mm.
Inlet Temperature: 210° C.
Detector Temperature: 220° C.
Oven program: 110° C. (1 min)→20° C./min→220° C. (10 min)
Run time: 22.5 min
Constant flow: 3 mL/see
Split ratio: 1:20
Injection volume: 1 mL

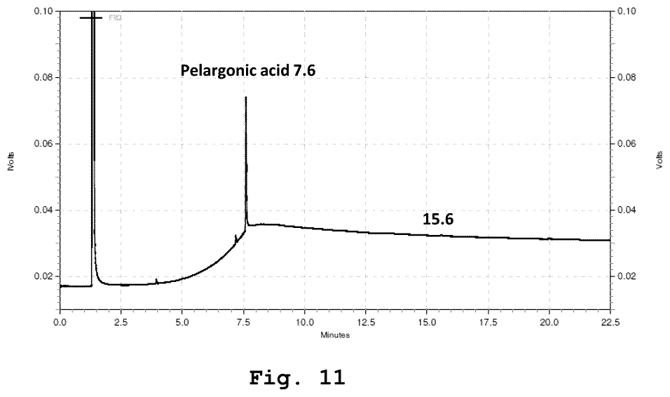

Chromatogram of 0.5 mg/mL Pelargonic Acid standard obtained with GC method is demonstrated on FIG. 11.

### Determination of Optimal Parameters of Reactions

For determination of optimal conditions of trioleate conversion, 20 g olive oil and 2.8 g KMnO₄ were mixed.

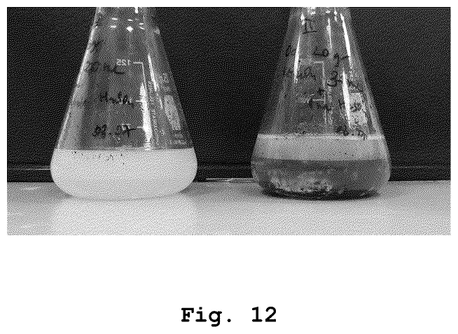

Olive oil was placed in the flask, KMnO₄ is added as a solid with small amount of water. The mixture was heated to 60-80° C. After about 15 min, 35% H₂O₂ was added, and flask was put into refrigerator. After the addition of H₂O₂, volume of reaction mixture increased at least twice, and intensive bubbling was observed. After a while, the reaction mixture brightened, and two liquid layers were observed. The products of the reaction are water-soluble. If KMnO₄ is still in excess in respect to H₂O₂, a pink color of KMnO4 in water phase and unreacted KMnO₄ in the bottom of the flask is observed as demonstrated on FIG. 12.

When H₂O₂ was added together with KMnO₄, HPLC analysis showed that the amount of trioleate remained the same and Pelargonic Acid was not observed.

When H₂O₂ was not added, HPLC results regarding Trioleate and Pelargonic acid remained approximately the same. However, in this case, separation between two phases was difficult to achieve due to the high content of unreacted potassium permanganate and formed MnO₂. Clear separation was achieved with filtration of solid phase out. The analysis of trioleate showed only insignificant decrease after the reaction. Assay of Trioleate in Olive oil dropped from 28% to 20%. Assay of Pelargonic acid was even lower, it did not correspond to 8% of trioleate conversion.

Ratios: 1:1; 1:2; 1:4 ratio values and found that equal ratio between moles of trioleate and moles of KMnO₄ multiplied by 3 is enough. No significant difference between reaction temperature within the range 20-80° C. and time of exposure (15-45 min). Although Triolein e content in olive oil decreased, only about 0.15% of pelargonic acid formed. This indicates that the reaction of trioleate with Potassium Permanganate led mainly to hydroxylation of double bond rather than cleavage, e.g., only small amount of Triolein e reacted to produce Triazelate and pelargonic acid.

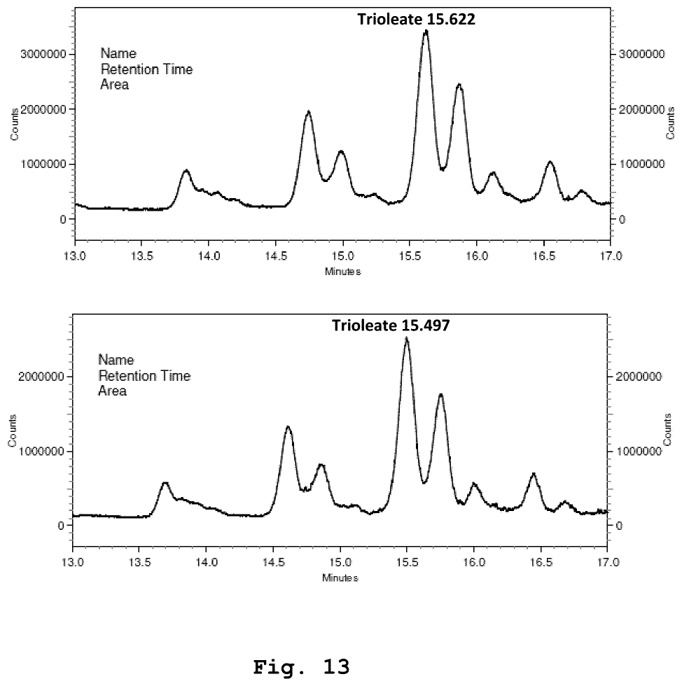

Therefore, despite low conversion of Trioleate, reaction is easy to perform, and the products are liquid and well-separated. It should be noted that there is a significant change of color of olive oil after the addition of hydrogen peroxide from deep yellow green to yellowish, and, as represented on FIG. 13, the only difference of chromatogram of olive oil before and after reaction is intensity.

To increase the degree of conversion of Triolein to Triazelate, Olive Oil was continuously with excess of KMnO₄ for 3 hours at RT.

The reaction was carried out as follows:

40 g of saturated KMnO₄ solution was mixed with 20 g of Olive oil and mixed for 3 h. After 3 h, 1 mL H₂SO₄conc was added and mixed for an additional hour.

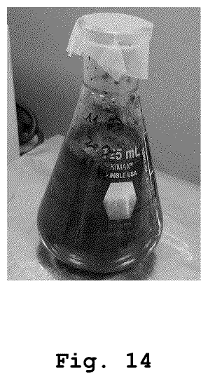

The result of the process was marsh-like black-brown viscous liquid as shown on FIG. 14.

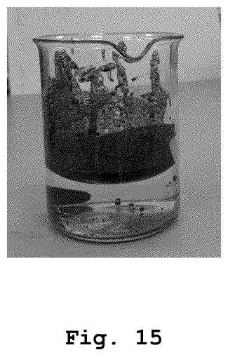

Upon addition of large amount of water, it was apparent that that MnO₂ is in a dark oil phase, as shown on FIG. 15.

Filtration was difficult due to the high amount of sticky MnO₂. In addition, oil phase and water phase can be separated via centrifugation. Oil phase remained in very small amount—about 1 g.

Addition of H₂O₂ resulted in bubbling for few seconds indicating that part of permanganate was still not consumed.

To increase exposure of olive oil to permanganate, the reaction was carried out in the presence of Brij 35, to combine oil and water phases thus making olive oil exposed to KMnO₄.

Visually, the results were similar however, sample with Brij 35 appeared more liquid and was easily filtered. In sample with Brij 35, MnO₂ was distributed into the whole volume of the flask.

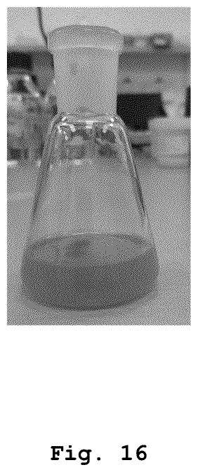

After filtration, a single phase was observed as shown on FIG. 16. Full clearance of the liquid is achieved with centrifugation.

Liquid formed in the reaction was not miscible with Hexane or in methanol, but it was miscible with water.

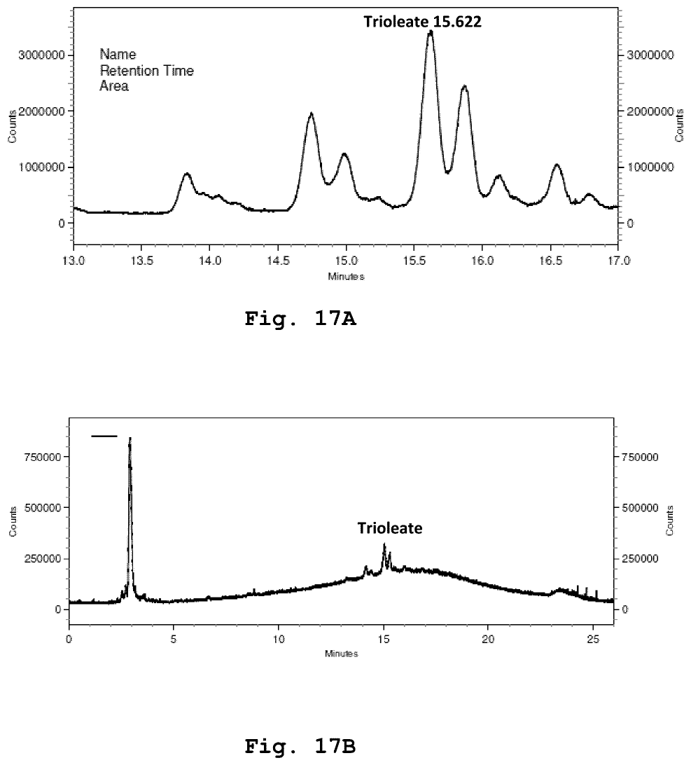

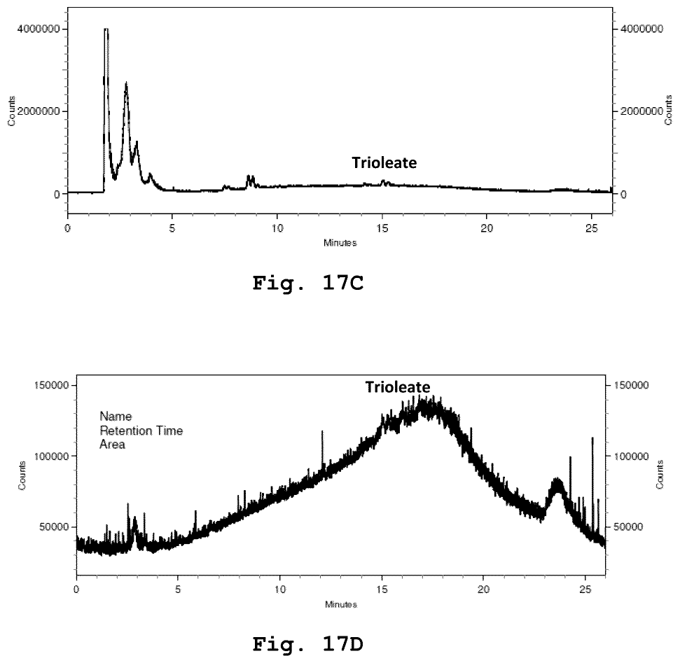

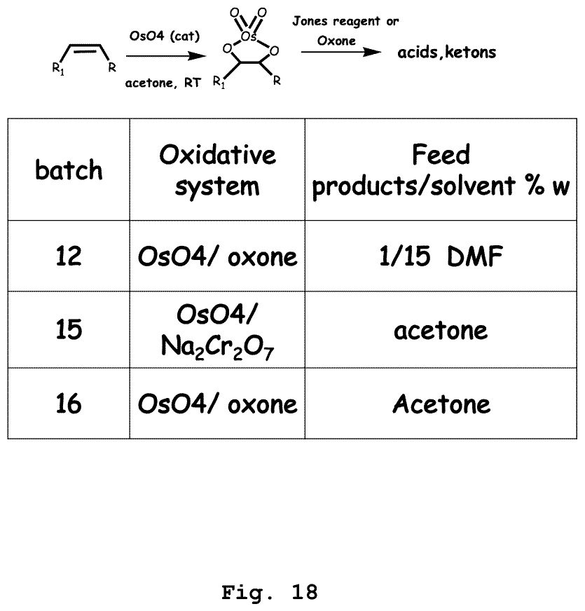

As shown on FIGS. 17A-17D, trioleate disappeared because of the reaction. FIGS. 17A and 17B show the trioleate peak before and after the reaction, respectively. It is seen in the chromatogram in methanol extraction in FIG. 17C that more hydrophilic products were formed, while new peaks appeared. FIG. 17D shows preparation in Acetonitrile: Chloroform (90:10).

Pelargonic Acid was detected in all samples, but the amount of Pelargonic Acid didn't correlate with the drop of Trioleate.

### Conclusions

H₂O₂ is not involved in the conversion of Trioleate to Triazelate.
Only KMnO₄ is necessary for reaction.
Degree of Trioleate conversion depends on the reaction time.
Trioleate was fully conversed with prolonged exposure.
Presence of surfactant in reaction medium enhances the reaction until full disappearance of oil phase.

### Test for Triazelate

We repeated the preparation of Triazelate using larger amounts of olive oil.

The Olive oil was prepared using three methods:
KMnO₄ for short period followed by addition of H₂O₂
KMnO₄ for long period
KMnO₄ in the presence of surfactant Brij 35
There are five samples were prepared:

TABLE 5
Visual specifications of in process control of TriAza process.

| Sample # | Phase | Procedure | Visual observation |
|---|---|---|---|
| 1 | Upper | 100 g of Olive oil + | After addition of |
| | (oil | 150 g KMnO₄ saturated | H₂O₂, excess of |
| | phase) | solution (7 g/L) + 5 mL | KMnO₄ disappeared. |
| 2 | Under | H₂SO₄. Mixed for 45 | Two bright phases |
| | (aqueous | min at 40° C. | are observed. |
| | phase) | H₂O₂ (35%) was added. | |
| 3 | Upper | 100 g of Olive oil + | Phases can be |
| | (oil | 150 g KMnO₄ saturated | observed only after |
| | phase) | solution (7 g/L) + | filtration |
| 4 | Under | 5 mL H₂SO₄. Mixed for | (separation from |
| | (aqueous | 3 h at 40° C. | MnO₂). |
| | phase) | | |
| 5 | One | 100 g Olive Oil + 150 g | Only one phase |
| | phase | KMnO4 + 5 g Brij 35. | was observed. |
| | | Mixed for 3 h at 40° C. | |

Since standard Triazelate is unavailable, the only way to conclude about the presence of Triazelate is MS.

All samples were run on GC-MS under the same conditions.

Azelaic Acid was observed only in water phase after the long treatment (sample 4). Pelargonic acid was not observed in this sample. However, Pelargonic Acid is present to a significant extent in oil phase.

Sample with Brij 35 has high concentration of Pelargonic Acid, chromatogram shows many peaks attributed to polyethylene glycol.

None of the samples showed peak with molecular weight 602. Therefore, it is unclear if Triazelate is formed or just cannot be detected. Table 6 summarizes significant peaks in GC-MS chromatograms.

TABLE 6
End products detected as part of identity specifications of TriAza process production.

| | RT | | | | | | | |
|---|---|---|---|---|---|---|---|---|
| | RT | RT | RT | RT | RT | RT | RT | RT |
| | 9.67 | 10.38 | 11.14 | 12.54 | 13.62 | 15.4 | 16.1 | 16.23 |
| | name | | | | | | | |
| | pelargonic | capric | pelargonic | | azelaic | ethyl | oleic | ethyl |
| | aldehyde | acid | acid | Dodecanol | acid | palmitate | acid | oleate |
| sample 1 | yes | no | yes | no | no | yes | yes | yes |
| sample 2 | yes | no | yes | no | no | yes | yes | yes |
| sample 3 | yes | no | yes | no | no | yes | yes | yes |
| sample 4 | no | no | no | no | yes | no | yes | no |
| sample 5 | no | yes | yes | yes | no | no | no | no |

Finally, Triolein according to the method and peak of Triolein was not found. Further, we run the same samples with HPLC-MS, without HPLC column, just making a scan of molecular weights in sample.

In diluent Acetonitrile/Chloroform, peaks of Triolein and Triazelate were not observed.

After addition of Ammonium Formate in diluent, strong Trioleate signal appeared with mass +903 indicating +18 of ammonium ion. Triazelate appeared as a peak with a mass +603 indicating that ammonium ion is not required.

Triazelate was observed in all samples, in water and oil phases.

No peaks attributed to mono- and di-azelates were observed.
Trioleate was fully converted to more hydrophilic products under prolonged reaction with KMnO₄.
The presence of Triazelate and Pelargonic acid in reaction products was proved with HPLC-MS method.

### Example 10: Ozonolysis and Synthesis Examples

The synthesis consists of three steps only, making it simple and optimized as ecologically safe and cost effective:
(I) OLIVE OIL→(2) OXIDATION/OZONOLYZIZ→(3) SOAP-LIKE FORMULATION (10%-20% of 3A)

This synthesis was performed by ozonolysis, an ecology-friendly method utilizing Triolein, a natural material, as a substrate. Ozonolysis relies upon ozone-mediated breakage of Triolein-containing double bounds that is followed by a production of COOH groups (Scheme 1). This method includes two steps: 1. exposure of Triolein to the mixture of ozone and oxygen (in a solvent) and 2. oxidation of first reaction products by oxygen in acidic environment.

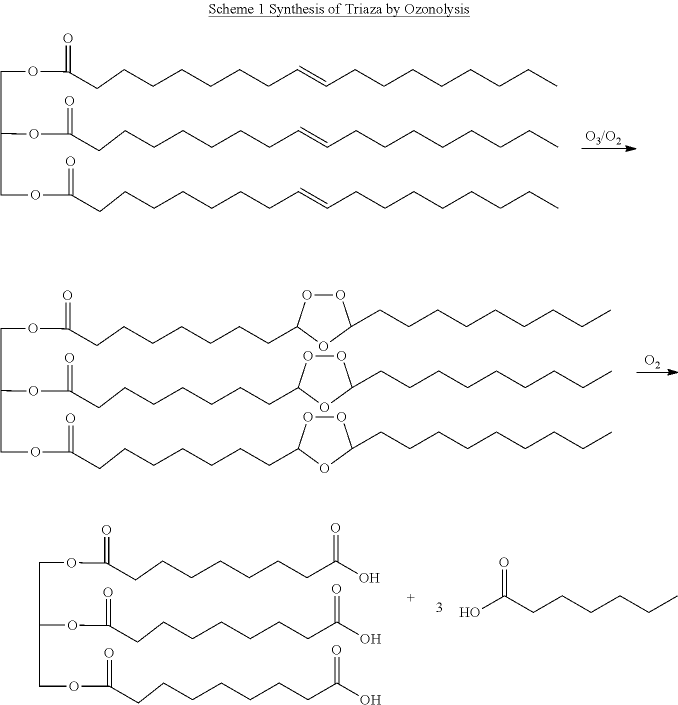

Transesterification is an additional method of current invention, is a structural lipid modification method of preparation of TriAza- to prepare TriAza-tributyrin, Triolein, or other natural based selected triglyceride comprising the at least one unsaturated or middle chain fatty acid as a constituent fatty acid may be subjected to transesterification: in such reaction lipase enzymatic method may be used or a catalytic method based on chemical catalyst to be used in ester interchange may be a chemical catalyst. In ester interchange using a chemical catalyst, the constituent fatty acids will be bonded at random positions. The applicable chemical catalyst may be exemplified by alkali metal hydroxides such as lithium hydroxide, sodium hydroxide and potassium hydroxide, or alkali metal alkoxides such as lithium methoxide and sodium methoxide. In the case of using a chemical catalyst, about 0.1-2 wt % of the catalyst may first be added to the mixed oil (natural triglyceride and azelaic acid, or any R selected from R description) consisting of the triglyceride, and the resulting mixture is subjected to reaction under stirring at an atmospheric or reduced pressure for 3-120 minutes at 50-270.degree. C. By performing ordinary purification steps such as washing with water, drying, decoloring, and deodorizing, the end-product can be obtained.

Substrate (Triaza) for lipase-mediated activity was synthesized. This synthesis was performed by ozonolysis, an ecology-friendly method utilizing Triolein, a natural material, as a substrate. Ozonolysis relies upon ozone-mediated breakage of Triolein-containing double bounds that is followed by a production of COOH groups (Scheme 1). This method includes two steps: 1. exposure of Triolein to the mixture of ozone and oxygen (in a solvent) and 2. oxidation of first reaction products by oxygen in acidic environment.

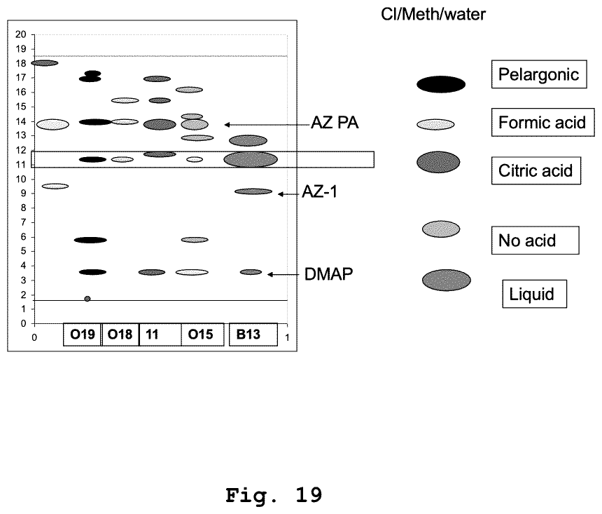

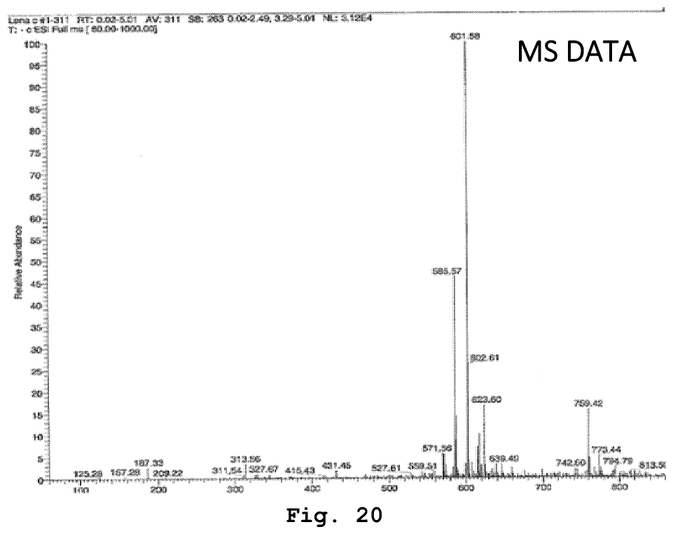

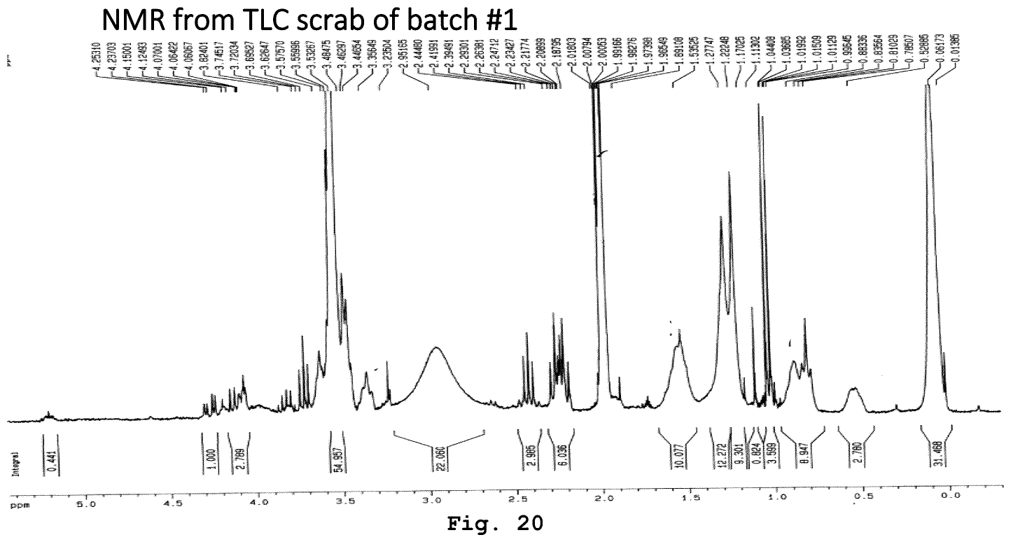

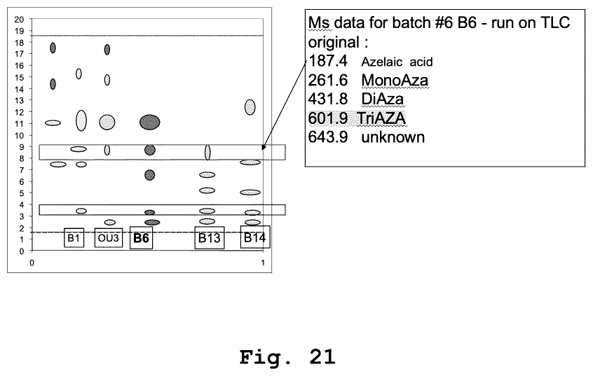

The parameters that were variably used are solvents (dichlormethane, pelargonic acid, formic acid, water, n-propylacetate), feeding acids (pelargonic, formic, acetic, citric acids), and reaction time. In a result 17 batches were produced that were analyzed by thin-layer chromatography (TLC), nuclear-magnetic resonance (NMR), and mass spectroscopy (MS). To confirm the results produced by ozonolysis of Triolein, Triaza was synthesized by either oxidation of Triolein using OsO4 or reacting glycerol with azelaic acid (4 batches) FIGS. 18 to 21. The materials synthesized by these methods were analyzed by TLC along with the products of ozonolysis. Triaza synthesized by ozonolysis were subjected to MS, TLC, and NMR analysis. An analysis of molecular mass revealed that all synthesized batches contain mono-, di-, and triglycerides of Triaza family. In addition, Triolein, Triaza-containing aldehydes, diglyceride were identified with both OH and COOH termini, triglycerides with dimmers of azelaic acid, and pelargonic acid. To further confirm the presence of ozonides in batches 3H-NMR was performed. The analysis has revealed that none of the batches contained ozonides and all batches were almost free of molecules possessing double bounds. Since Triolein is the only double bound containing molecule, it was concluded that the substrate is not completely utilized in the reaction. Moreover, an absence of ozonides in the samples is evident to their destruction and producing modified forms of medium-chain triglycerides. For the detailed analysis of Triaza, the batches produced by ozonolysis were analyzed by TLC and compared to that synthesized by liquid synthesis. All samples were separated by normal phase chromatography and visualized by either general dye (primulin) or COOH-specific staining (BromCresol Green). It was found that most batches of ozonolysis contain 4-7 clear spots (Rf of the resulting products were identified and compared with known standards). Out of them, 3 major spots were visualized with BromCresol Green, suggesting presence of carboxyl ends. Among them, one spot represents pelargonic acid, hence two additional spots might correspond to Triaza molecules. Comparison of Rf of substances obtained by ozonolysis and liquid synthesis has revealed that among the COOH-containing spots two of them are common, i.e., they might represent Triaza. Altogether, we have concluded that using ozonolysis of Triolein we succeeded in producing Triaza.

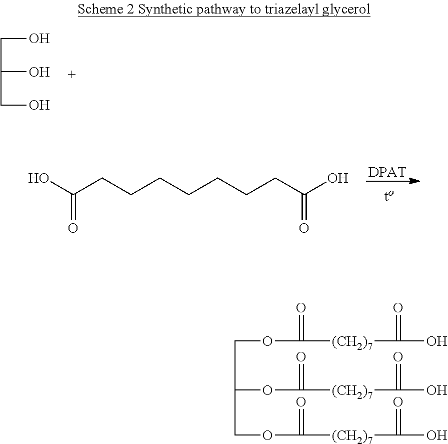

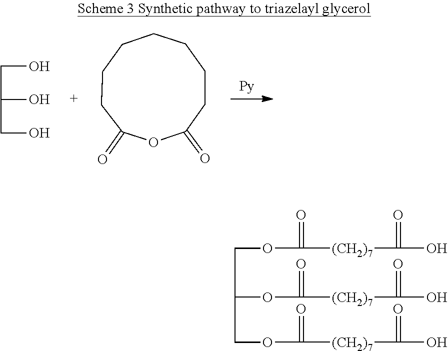

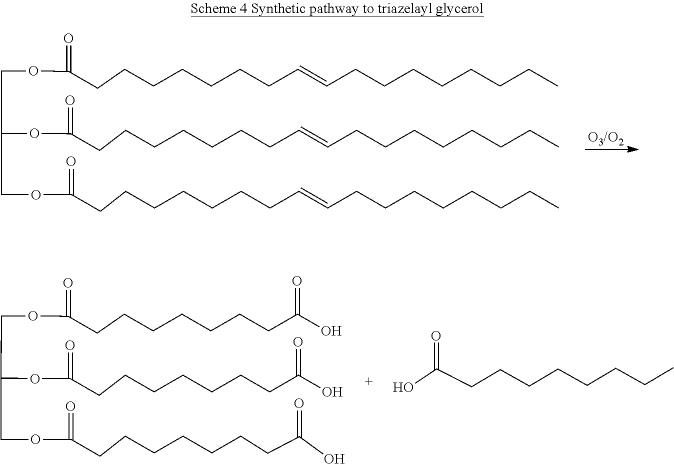

### Example 11: Selection of Process Conditions to Produce TriAza with Green Surfactants

The raw material Olive oil comprises up to 50% oleic acid. Oleic acid is present as free oleic acid, monooleate, di-oleate and tri-oleate. KMnO₄ added to olive oil cleaves double bond of oleic acid leading to formation of the following products: pelargonic and azelaic acid (followed by cleavage of oleic acid), monoazelate (followed by cleavage of monooleate), di-azelate (followed by cleavage of di-oleate) and tri-azelate (followed by cleavage of tri-oleate). An additional product of this reaction is MnO₂-precipitates of black powder. The reaction between olive oil and potassium permanganate under various conditions was performed. The results are summarized in Table 7:

TABLE 7
Results obtained under various conditions in the reaction
between olive oil and potassium permanganate.

| | | | | | | | QC | QC | QC |
|---|---|---|---|---|---|---|---|---|---|
| | | | | | | | Purity: | Identity: | Identity: |
| | | | | Time | | Visual | Trioleate | Pelargonic | TriAza |
| # | Surfactant | Citric | H2O2 | of mix | | Observation | HPLC | GC | MS |
| 1 | − | − | − | 20 | min | Two phases | + | + | + |
| 2 | − | − | − | 1 | h | Two phases | − | + | + |
| 3 | − | + | − | 20 | min | Two phases | + | + | + |
| 4 | − | + | − | 1 | h | Two phases | − | + | + |
| 5 | − | + | + | 20 | min | Two phases | + | + | + |
| 6 | Brij 35 | + | − | 1 | h | One | − | + | + |
| | | | | | | amphiphilic | | | |
| | | | | | | phase with | | | |
| | | | | | | flakes | | | |
| 7 | Sodium cocoyl | + | + | 1 | h | One phase | − | + | + |
| | glutamate | | | | | with flakes | | | |
| 8 | Sodium cocoyl | + | + | 20 | min | Two phases | − | + | + |
| | glutamate | | | | | | | | |
| 9 | Sodium lauroyl | + | + | 20 | min | Two phases | − | + | + |
| | sarcosinate | | | | | | | | |
| 10 | Sodium lauroyl | + | + | 1 | h | One | − | + | + |
| | sarcosinate | | | | | amphiphilic | | | |
| | | | | | | phase with | | | |
| | | | | | | flakes | | | |
| 11 | Sodium cocoyl | + | + | 1 | h | Two phases | + | + | + |
| | glutamate | | | | | | | | |
| 12 | Sodium cocoyl | + | + | 1 | h | One oil | + | + | + |
| | glutamate | | | | | phase | | | |

### Example 12: Alternative Method of Synthesis of Azelaic Acid Glyceryl Triester: Liquid Esterification Synthesis

A solution of 2.06 g of dicyclohexylcarbodiimide (DCC) in 15 ml (DCM) Dichloromethane was added very slowly (dropwise) to the solution of 0.22 g (2.5 mmol) of dry glycerol, 0.03 g (0.25 mmol, DMAP) N, N-dimethylaminopyridine, and 0.3 g (16 mmol) of azelaic acid dissolved in 4 ml of tetrahydrofuran (THF) in 18 ml d (DCM) dichloromethane. The resulting suspension was stirred overnight at 25° C. Dicyclohexyl urea (DCU) was filtered in vacuum and the solvent was evaporated under reduced pressure without heating. The residue was re-dissolved in THF and non-dissolved material (DCU) was separated. THF was evaporated to dryness until an oily residue was left in the flask. This oily residue solidified after lyophilizing. The resulting material was purified on preparative thin layer chromatography using chlorophorm/methanol/water (65:25:4, v/v/v) as the developing solvent.

### Results

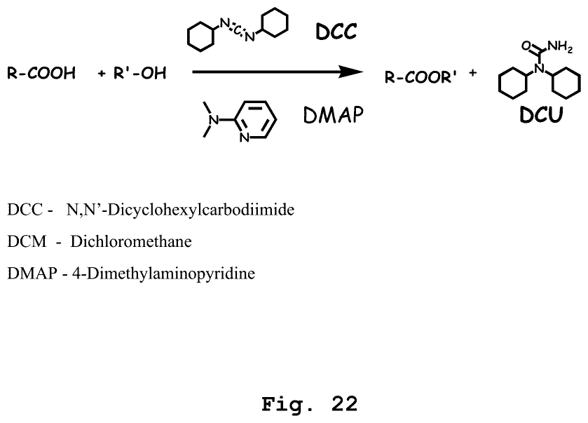

Yield: around 25-30%). NMR analysis of the resulting compound demonstrated relevant spectra). FIG. 22 shows the schematic pathway for this synthesis of azelaic acid glyceryl triester.

### Example 13: Alternative Method of Synthesis of Azelaic Acid Glyceryl Triester

In the example below, the goal was to obtain two major products: completely substituted glycerol, and mono-substituted glycerol. Two ratios of feeding products were used. An overloading of azelaic acid (batches 13, 14,17-4.3 mol instead of 3.0 mol) and underloading of azelaic acid in batch 20 (0.8 mol instead 1 mol), as depicted in Table 8:

TABLE 8
Ratios of feeding products glycerol and azelaic acid

| | Glycerol/Az | Feed |
|---|---|---|
| | acid | products/solvent |
| batch | [mol/mol] | [g/ml] |
| 13 | 1/4.3 | 1.7 g/54 ml |
| 14 | | 1.7 g/75 ml |
| 17 | | 1.7 g/43 ml |
| 20 | 1/0.8 | 0.5 g/37 ml |

Obtained results of transesterification using excess of glycerol resulted in highest yield of Triglycerol, Di Glycerol and Mono Glycerol, while using excess of azelaic acid resulted in the highest yield of triazelaine, as was proven by MasSpec and NMR spectra post TLC separation. The analyses revealed that absence of double bond, thus indicating full oxidation Triolein; and presence of glycerol backbone structures with acylated glycerol as the major component.

### Example 14

### Alternative Method of Synthesis of Azelaic Acid Glyceryl Triester.

The following procedure was carried out to enable a complete dissolution of azelaic acid. Starting components were mixed in equimolar concentrations in the DCM solvent and added dropwise to the mixture solution of DCC having the same solvent, as follows: Mixture 1:

185 μl glycerol (0.23 g, 2.5 mmol) was dried on molecular sieves4A and MgCl2. 26 ml DCM containing 1.65 g (8.7 mmol) azelaic acid and 5 ml THF was mixed in round-bottom 50 ml flask. 3 ml DMF was added to dissolve the solution. 2.06 g DCC was dissolved in 15 ml CH2CL2. 1.5 ml DCC solution was added dropwise. Then 185 μl (2 mM) glycerol was added. The rest of the DCC solution was then added. DCU began to precipitate during past ¼ of all DCC quantity. The mixture was allowed to stir for 3 hours and was transferred to 150 ml Erlenmeyer and incubated for 24 hours to obtain TriAza. The mixture was allowed to stir for 3 hours and was transferred to 150 ml Erlenmeyer and incubated for 24 hours to obtain TriAza.

### Example 15: Lipase Mediated Activity

The activity of C. acnes-associated and P. acnes-associated lipase was tested in the presence of triolein (a trigger of lipase activity). Bacteria was grown for 4 days in liquid medium. P. acnes after double passage on Chocolate plates (Novamed) were grown bacteria in anaerobic conditions at 37° C. for 1-2 days. The bacteria were Gram stained and the proper morphology of the bacteria was assured. Then, 1 O.D. 600 bacteria were resuspend in 1 ml of Thioglycolate medium (HyLabs) containing 50 μl triolein and grown in anaerobic conditions at 37° C. for 4 days. Following this incubation, 100 μl of emulsified substrate was added to the culture that was further maintained at 37° C. for over night. Afterwards, the supernatant was separated from the bacteria by 1 min centrifugation at 10000 rpm and subjected to lipid extraction using clorophorm:methanol (2:1). The lipids after nitrogen evaporation and resuspension in clorophorm:methanol (2:1) were separated on silica TLC plates in a development solution of clorophorm: methanol: water (65:25:4), and visualized with BromCresol Green (Sigma).

### Example 16: Triolein is a Sufficient Substrate for Lipase Producing Microorganisms on Example of Staph. Aureus

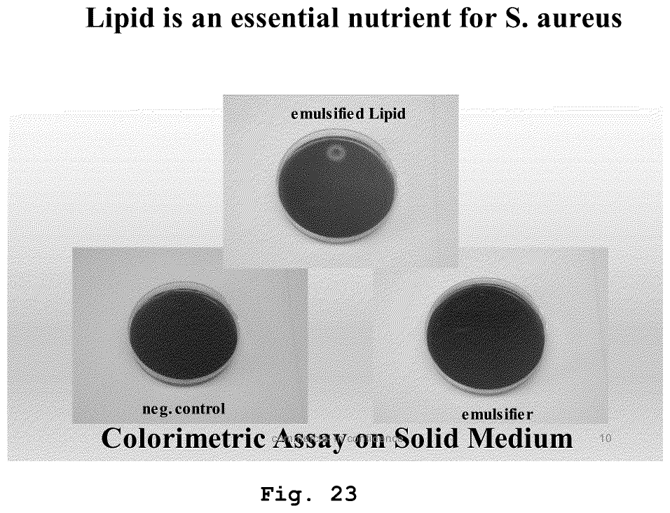

S. aureus bacteria was plated on the solid agar Petri dishes (1) nutrient free, (2) agar with spot with emulsified Triolein, and (3) plate with spot with emulsifier only. On the plates bacteria were incubated at 37° C. for 2 days. S. aureus growth was tested using colorimetric assay in the presence of emulsified triolein solid medium agar culture as shown in FIG. 23.

### Results

It appears that triglyceride (TG) is a sufficient and essential nutrient for skin microbiome, as shown in the plate containing emulsified triolein which allowed bacterial colonization, compared to lack of growth in the negative control or the plate containing only the emulsifier (FIG. 23). It is concluded that lipase-mediated hydrolysis of triolein generates free fatty acid as an essential energy source for growth of S. aureus.

### Example 17: C. Acnes-Associated Lipase Mediated Activity

To determine the affinity of a bacterial lipase to the active ingredient, the ability of the lipase to utilize TriAza as a substrate was tested. Natural emulsifier, gum acacia at the concentration of 5% was prepared in bacterial medium or in 0.1M citric/phosphate buffer (pH 7.5). The gum acacia solution was emulsified using water-bath sonicator for 15 min. Afterwards 100 mg lipid-based molecules were added to 400 μl of 5% gum acacia and sonicated. To test activity of C. acnes generated lipase, C. acnes suspension culture after double passage on Chocolate plates (Novamed) was maintained in anaerobic conditions at 37° C. for 2 days. The bacteria were Gram stained and the proper morphology of the bacteria was assured. Then, 1 O. D. 600 bacteria were resuspended in 1 ml of Thioglycolate medium (HyLabs) containing 50 mcl of TG and grown in anaerobic conditions at 37° C. for 4 days. Following the incubation, 100 mcl of emulsified substrate was added to the culture that was further maintained at 37° C. overnight. Afterwards, the supernatant was separated from the bacteria by 1 min centrifugation at 10000 rpm and subjected to lipid extraction using clorophorm: methanol (2:1). The lipids after nitrogen evaporation and resuspension in clorophorm:methanol (2:1) were separated on silica TLC plates in a development solution of clorophorm: methanol: water (65:25:4), and visualized with BromCresol Green (Sigma).

### Results

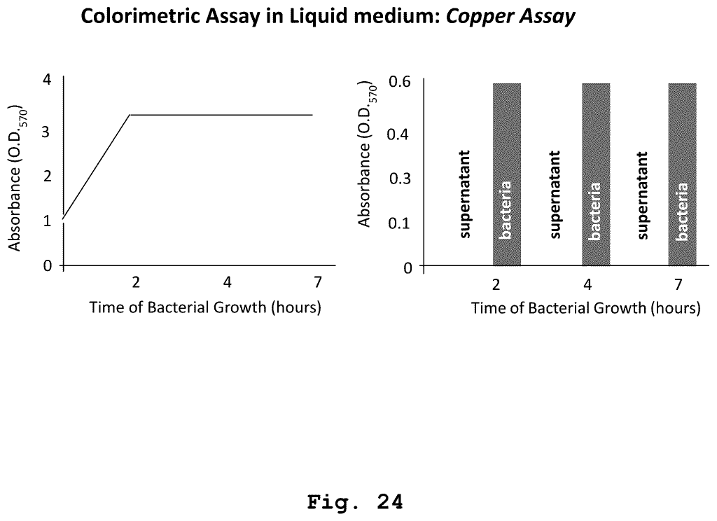

It was found that C. acnes exposed to TriAza produces a significant amount of azelaic acid as demonstrated in FIG. 24. These results imply that similarly to purified lipase from C. cepacia, C. acnes-associated lipase recognizes TriAza as an active substrate. Thus, TriAza might serve as a pro-drug that releases azelaic acid in response to activity of the bacteria-produced virulent lipase enzyme.

### Example 18: Selection of Methods for QC-Quality Control-Functional Potency: Testing Minimal Inhibitory Concentration to P. acnes by Microdilution

P. acnes after double passage on Chocolate plates (Novamed) were grown bacteria in anaerobic conditions at 37° C. for 1-2 days. The bacteria were Gram stained and the proper morphology of the bacteria was assured. Then, bacteria were resuspend in Thioglycolate medium (HyLabs) at concentration of 0.02 O.D.₆₀₀. Meanwhile, ELISA plates containing at the first raw 200 ul of undiluted tested material were prepared. The rest wells were filled with 100 ul of Thioglycolate medium and 11 serial ½ dilutions by transferring 100 ul of undiluted material was performed. Once the ELISA plate was ready, 100 ul of prepared bacterial culture was added. Bacteria alone and medium w/o bacteria were used as standard controls. The plates were incubated in anaerobic conditions at 37° C. for 2 days. Minimal inhibitory concentration was determined visually and spectrophotometrically, while minimal bactericidal concentration was determined after plating of the bacteria on solid medium (Chocolate plates). On the plates bacteria were incubated in anaerobic conditions at 37° C. for 2 days and the number of appeared colonies were counted manually. Specification for positive functional potency for batch acceptance for the TriAza batches tested are selected in range of antimicrobial activity-MIC between 0,25-1 mM.

### Example 19: C9 OCFA/TG9-TriAza Mixtures Generation

Odd numbered fatty acids are found in small amounts acylated to various sphingolipids where they have unique properties and functions. Phospholipids of azelaic acid, cerebrosides, sphingolipids as natural metabolites of odd chain fatty acids are described in prior art.

No use of external compositions of mixtures comprising triglycerides, diglycerides, monoglycerides and mixtures enriched with triacylglycerols of azelaic acid and other C9 OCFA for use in longevity multifunctional food and topical formulations were described before.

Suprisingly, the disclosed composition can be enriched with the precursors of odd chain fatty acids. OCFA can endogenously bio-synthetized from precursors short chain FAs (e.g. tripropionate, propionic acid, succinate and derivativs of thereof) to long chain OCFA (C15; C17-ceramides) by internal elongases and other enzymatic machinery as in mammalian host and also, putatively, with help of microbiome.

TRiAza multifunctional glycerol conjugates or C9 MOCT/OCFA mixtures may comprise additional precursors or/and derivatives of OCFA (as exemplified in Table 9 below) and also derivatives, such as esters, dicarboxylic (e.g. brassylic acid), conjugated, hydroxy and branched isomers and glycerol's. Intracellular fatty acid elongase enzymes generate C9 OCFA from OCFA precursor with C 3-7 and derivative of OCFA can be derived/generated by elongases from C9 towards C11; C13 (brassylic), C15, C17 etc. up to very long chain saturated-OCFA (Table 9A). Commercialy available today C9 derivatives linear and branched are substrate for esterases and will form C9 FA post hydrolytic cleavage by intacellular esterase/hydrolase enzymes, same relevant for diesters of azelaic acid dicarboxylic precursors and diester derivatives (Table 9B).

TABLE 9
Precursors or/and derivatives of OCFA

| A. C9 FA Precursors | |
|---|---|
| and Derivatives/ | B. Derivatives/precursorslinear |
| Carbon number | and branced nonanoates esters, |
| Systematic Name | diesters of C9 FA |
| C3-Propanoic acid | ISONONYL ISONONANOATE |
| C5-Pentanoic acid | CETEARYL NONANOATE |
| C7-Heptanoic acid | ETHYLHEXYL PELARGONATE |
| C9-Nonanoic acid | PROPANEDIOL DIPELARGONATE |
| C11-Undecanoic acid | CETYL STEARYL ISONONANOATE |
| C13-Tridecanoic acid | NEOPENTYL GLYCOL |
| | DIISONONANOATE |
| C15-Pentadecanoic acid | PENTAERYTHRITYL |
| | TETRAPELARGONATE |
| C17-Heptadecanoic acid | DIETHYL AZELATE |

TriAza can be obtained by oxidation of vegetable oils or animal fats as raw material enriched with triglycerides (TGs), or purified TG that comprise fatty acid-FA with n-9 (omega-9) unsaturation bond, or TG of C20 (n-11), whereas during oxidative cleavage, essentially at acidic conditions, i.e. without hydrolysis, TriAza examples of relevant FAs depicted below:

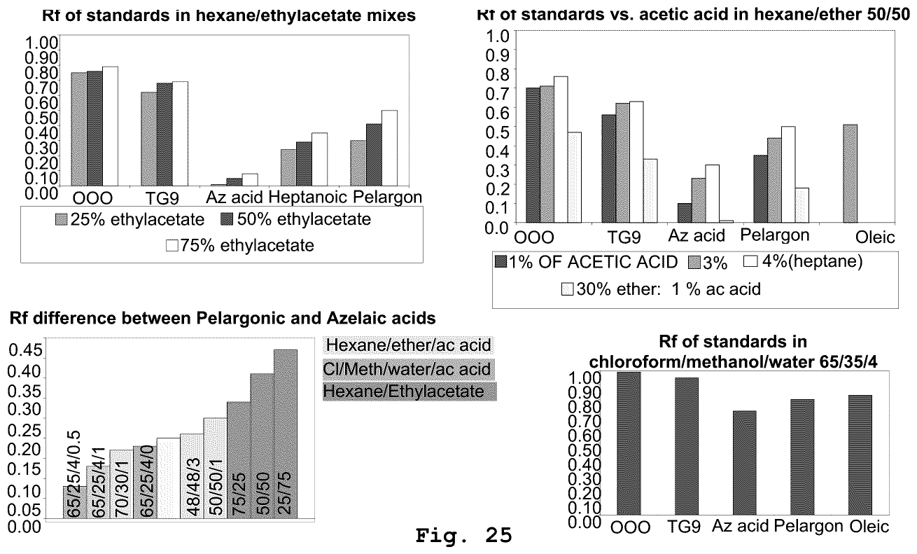

Analytical QC characterization. Thin layer chromatography (TLC) assays were tested with different running solvents for better separation of pair triacylglycerols—FFA and FFA-dicarboxylic acids. The best results were obtained with primulin. Bromcresol green was chosen for distinction between triglycerides and fatty acids: 0.05-1% solution Rhodamine B in ethanol or 2% solution in water following with spraying 10N KOH; 0.01% (w/v) primulin solution in acetone-water (60:40, v/v) with UV detection; 0.2% 2,7-dichlorofluorescein in methanol; 0.3 g Bromcresol green in mix Methanol/water (80° ml/200) with 8 drops of 30% NaOH; Sensitivity of coloring: about 1 μl of 100 mM solutions. Representative results of qualitative and quantitative control analytical results, represented in FIG. 25, characterize relative recovery of C9 Triaza derivatives of ozonolysis reaction including: 000-ozonides, AzaA-azelaic acid, Pelargonic acid/PA, and TriAza-TG9-derivatives.

### Results

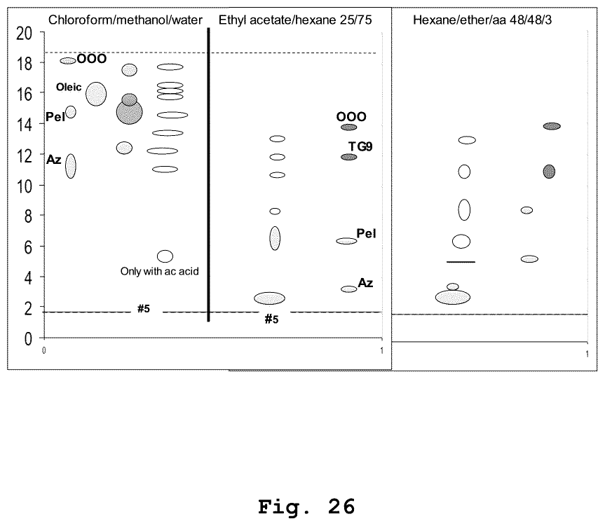

TLC spot distributionwere analysed in different feeding acid and methods and selected based on chloroform/methanol/water best separation ability. The chromatography, shown in FIG. 26 indicates that ozonolysis feed method with formic acid results (grey spots) in three end products of the reaction only: TriAza-TG9-(proven by MS and NMR analysis), PA-pelargonic by product and single ozonide band as non-utilized raw material. Esterification method-liquid synthesis resulted in highest yield of TriAza and di-Aza as by product, with underutilized azelaic acid (utilized in oversaturation) and DMAP reagent as by products. TriAza mix contained TriAza and Pelargonic acid recovered for MS analysis and chromatography by various analytical methods at various ratio as shown below.

MS were measured by LC/MS (Finnigan LCQ™ DUO, USA) in different modes. The LCQDUO MS detector uses quadrupole ion trap mass analyzer with an ion source external to the mass analyzer. All data are presented in Table 10 below:

TABLE 10
MS-MS Mass-spectroscopy analytical results.

| | |
|---|---|
| I | 1081 |

| OOO | 885.4 | | 886.4 | 885.3 | | | | | | | 885.3 |
|---|---|---|---|---|---|---|---|---|---|---|---|
| G | | 778 | | | 773.4 | | 773.5 | | 778.4 | | 778.5 |
| F | | 759 | 756 | 757.2 | 759.4 | | 759.4 | | | | 759.4 |
| E | | | | | 742.6 | | 745.5 | | 745.4 | | |
| TRIAZA | 602.7 | | | | 602 | 603 | 602 | 603 | 603.5 | 604.5 | 603.5 |

| | | | | | | |
|---|---|---|---|---|---|---|
| D | 586 | 581.3 | 584.5 | 585.5 | 585.6 | 585.7 |

| | | | | | | | | |
|---|---|---|---|---|---|---|---|---|
| C | 570 | 564.9 | 559 | 572 | 570 | 572 | 569.7 | 569.7 |

| B | | 556 | | | 555.6 | 555.7 | 553.6 | | |
|---|---|---|---|---|---|---|---|---|---|
| DiAza | 432.5 | | 443.2 | 431.5 | 431.6 | | | | |
| A | | 416 | | 415.4 | 415.5 | 415.5 | 415 | 416 | 417 |
| MonoAza | 262.3 | | | | 261.4 | | | | |

| | | | | |
|---|---|---|---|---|
| Azel | 188.2 | 188.4 | 187.3 | 187.3 |

| | | | | | |
|---|---|---|---|---|---|
| Pelargonic | 158.24 | 157.1 | 157.3 | 157.3 | 177.3 |

| | | | | | |
|---|---|---|---|---|---|
| 1.21 E3 | 4.14 E2 | 3.12 E4 | 6.28 E4 | 4.33 E5 | 9.23 E4 |
| AV 309 | AV 13 | AV 311 | AV 479 | AV 30 | AV 30 |
| 0.02-4.93 | 0.78-1.02 | 0.02-5.01 | 0.03-7.73 | 0.59-1.07 | 1.16-1.66 |
| −c | −p | −c | −c | +p | +p |

| | | | | | |
|---|---|---|---|---|---|
| Batch | OOO | OOO | #1 | #2 | #3 |

| I | | | | | | | | | | | |
|---|---|---|---|---|---|---|---|---|---|---|---|
| OOO | 885.4 | | | 889.1 | | | | | | 881.8 | |
| G | | | | | | 778.3 | | | | | |
| F | | | | 761.3 | | | | | | 759.3 | |
| E | | 731.3 | | 747.3 | | 743.5 | | | | | |
| TRIAZA | 602.7 | 603.5 | | 603.5 | 604.5 | | | | | 601.6 | 601.7 |
| D | | 585.6 | | 585.6 | | | | | | 585.5 | 585.6 |
| C | | 569.7 | | 569.7 | | 569.8 | | | | | 569.5 |
| B | | 555.8 | | 555.8 | | 555.9 | | | | | |
| DiAza | 432.5 | | | 429.6 | | 429.4 | | | | | 431.1 |
| A | | 416 | 416 | 415.6 | | 415.7 | | | | | |
| MonoAza | 262.3 | 257.5 | | 259.3 | | 258 | 259 | 260 | 261 | | |
| Azel | 188.2 | | | 187.3 | | | | | | | |
| Pelargonic | 158.24 | | | | | | | | | | |

| | | | |
|---|---|---|---|
| 3.14 E5 | 2.77 E5 | 3.57E+03 | 1.33E+03 |
| AV 48 | AV 27 | AV19 | AV 24 |

| | | | | |
|---|---|---|---|---|
| 0.22-0.99 | 0.21-0.64 | 0.53-1.62 | 0.26-0.66 | 0.49-0.93 |
| +p | +p | +p | −p | −p |

| | | | |
|---|---|---|---|
| Batch | #4 | #5 | #6 |

| I | | 1203 | 1226.4 | | 1204.1 | 1203.7 | | 1203.7 |
|---|---|---|---|---|---|---|---|---|
| OOO | 885.4 | 988 | | | | | | 884.1 |
| G | | | | | | | | |
| F | | 757.9 | | | 758.1 | | | |
| E | | | 744 | 744.9 | | 743.6 | | 743.8 |
| TRIAZA | 602.7 | 601.9 | 602.4 | | 602.6 | 602 | 604.3 | 602.5 |
| D | | | 586.3 | | 588.5 | 588.7 | | 588.6 |
| C | | | 574.1 | | 574.3 | 574.6 | | 574.5 |
| B | | 558 | | | | | | |
| DiAza | 432.5 | 431.8 | 431.9 | | | 432.2 | | 432 |
| A | | | | | 413.9 | 430.7 | | |
| MonoAza | 262.3 | 261.6 | 263.2 | | 263.5 | 261.9 | | 261.4 |
| Azel | 188.2 | 187.4 | 187.6 | | 189.1 | 187.8 | | 187.7 |
| Pelargonic | 158.24 | | | | | | | |

| | | | | |
|---|---|---|---|---|
| 5.91 E4 | 6.28 E4 | 6.28 E4 | 1.59 E4 | 1.39 E4 |
| AV 180 | AV 479 | AV 479 | AV 8 | |
| 2.06-5.85 | 2.2-3.2 | 0.88-1.92 | 1.11-1.38 | 1.11-2.47 |
| −p | −p | −p | −p | AV 34 |

| | | | | | |
|---|---|---|---|---|---|
| Batch | O19 | 1 hour | O19 | 3 hour | O18 |

Besides of triolein, diglycerols of oleic and linoleic acids also produced Mm similar to TriAza by positive-ion APCI mass spectra (Scheme 5) and dioleyl glycerol produce characteristic ions with mass 603.6. Additional, mass spectra of triolein oxidation products included hydroperoxides, epoxides and ketones, analyzed by atmospheric pressure chemical ionization (APCI) mass spectrometric detection, produce Mm 601.6-603.6 for all samples.

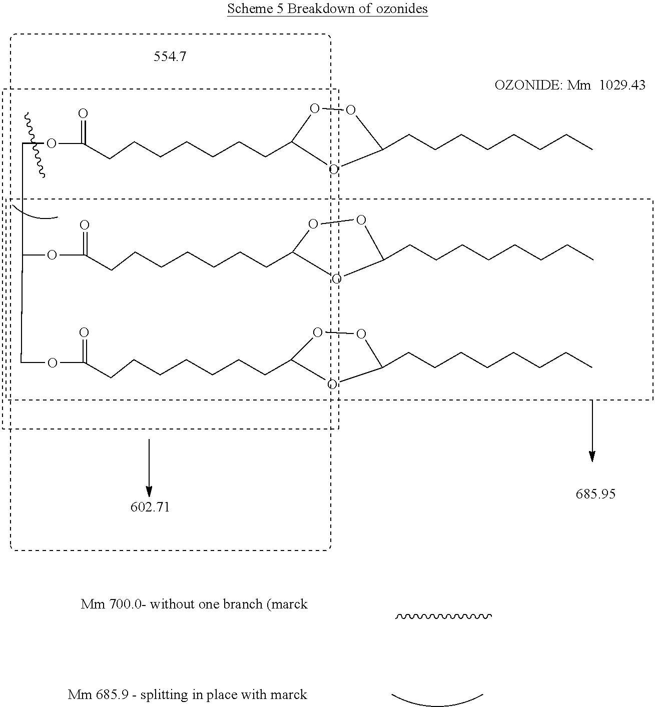 [SMILES: CCCCCCCCC1OOC(CCCCCCCC(=O)OCC(COC(=O)CCCCCCCC2OOC(CCCCCCCC)O2)OC(=O)CCCCCCCC2OOC(CCCCCCCC)O2)O1]

All spectra were compared to each other and the representative peaks were emphasized and summarized in Table 11 bellow:

TABLE 11
Mass- spectrometry observed for various substances

| Substance | Mm | Mm observed | Possible structure |
|---|---|---|---|
| Pelargonic | 158.24 | 157.3 | |
| C9-nonanoic | | | |
| Azelaic | 188.22 | 187.3 | |
| C9-dicarboxylic | | | |
| Mono-glycerol-Aza | 262.3 | 261.6 | |
| A-diglycerol C9 derivative 1 | 416.5 | 415.5 | 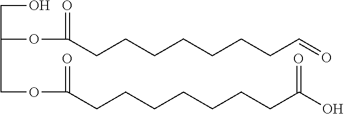 [SMILES: O=CCCCCCCCC(=O)OC(CO)COC(=O)CCCCCCCC(=O)O] |
| DiAza-diglycerol of | 432.51 | 431.6 | |
| azelaic acid | | | |
| B-TriAza derivative- keto | 554.7 | 555.8 | 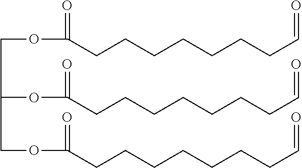 [SMILES: O=CCCCCCCCC(=O)OCC(COC(=O)CCCCCCCC=O)OC(=O)CCCCCCCC=O] |
| C TriAza derivative- mixed | 570.7 | 569.7 | 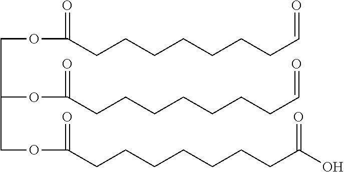 [SMILES: O=CCCCCCCCC(=O)OCC(COC(=O)CCCCCCCC(=O)O)OC(=O)CCCCCCCC=O] |
| D TriAza derivative mixed | 586.7 | 585.6 | 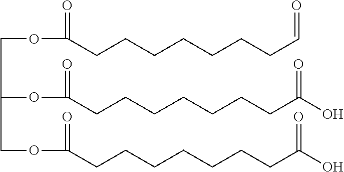 [SMILES: O=CCCCCCCCC(=O)OCC(COC(=O)CCCCCCCC(=O)O)OC(=O)CCCCCCCC(=O)O] |
| TriAza | 602.71 | 601.6 | 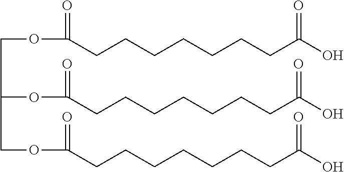 [SMILES: O=C(O)CCCCCCCC(=O)OCC(COC(=O)CCCCCCCC(=O)O)OC(=O)CCCCCCCC(=O)O] |
| F-di-olein derivative | 760.2 | 759 | 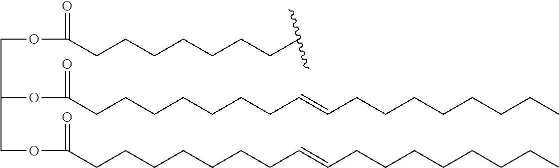 [SMILES: *CCCCCCCC(=O)OCC(COC(=O)CCCCCCC/C=C/CCCCCCCC)OC(=O)CCCCCCC/C=C/CCCCCCCC] |
| OOO | 885.4 | 884.1 | Ozonide -OOO not oxidized |
| I | | 1081 | Dimer of Mm 601.6 |
| E-ozonide derivative | 744.9 | 743.8-747 | 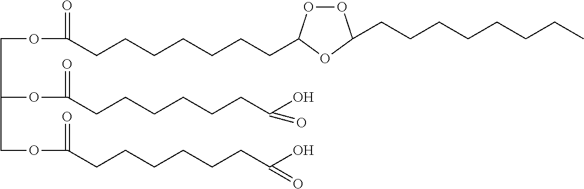 [SMILES: CCCCCCCCC1OOC(CCCCCCCC(=O)OCC(COC(=O)CCCCCCC(=O)O)OC(=O)CCCCCCC(=O)O)O1] |

### Example 20: Esterification Synthesis of TriAza as a Mixture of 2:1—Ratio of Azelaic Acid-Aza: Pelargonic Acid (PA)

A solution of DCC (725 mg, 3.5 mmol) and DMAP (430 mg, 3.5 mmol) in methylene chloride (10 ml) was added to a solution of glycerol (200 mg, 2.2 mmol), Aza (95%, 610 mg, 2.2 mmol) and PA (99%, 310 mg, 1.1 mmol) in methylene chloride (40 ml) at room temperature under nitrogen. As the reaction proceeded a precipitate dicyclohexylurea formed. After 5 h hexane (50 ml) was added to precipitate more dicyclohexylurea and the reaction was filtered and concentrated to dryness. Purification by flash chromatography (5% ethyl acetate/hexane) yielded the pure triglycerides as a colorless oil.

### Example 21: Esterification Synthesis of TriAza as a Mixture of 1:2 Ratio of—Pentadecanoic Acid-C15:Aza

A solution of DCC (725 mg, 3.5 mmol) and DMAP (430 mg, 3.5 mmol) in methylene chloride (10 ml) was added to a solution of glycerol (200 mg, 2.2 mmol), Aza (98%, 305 mg, 1.1 mmol) and C15 (99%, 620 mg, 2.2 mmol) in methylene chloride (40 ml) at room temperature under nitrogen. As the reaction proceeded a precipitate dicyclohexylurea formed. After 5 h hexane (50 ml) was added to precipitate more dicyclohexylurea and the reaction was filtered and concentrated to dryness. Purification by flash chromatography (5% ethyl acetate/hexane) yielded the pure triglycerides as a colorless oil.

### Example 22: Esterification Synthesis of TriAza as a Mixture 2:1 Ratio of Aza:RetA-Retinoic Acid

A mixture of glycerol (200 mg, 2.2 mmol), Aza (98%, 610 mg, 2.2 mmol), RetA (99%, 305 mmol, 1.1 mmol) and p-toluene sulfonic acid (20 mg) were heated at 140° C. for 5 h under a stream of nitrogen. The reaction was cooled and purified by flash chromatography (5% ethyl acetate/hexane) to yield the pure triglycerides as a colorless oil.

### Example 23: Esterification Synthesis of TriAza as a Mixture 1:2 Ratio of Aza:BrA-Brassylic C13 Dicarboxylic Acid

Notably triglycerol of C13-brassylic acid classical oxidative cleavage of olefinic compounds with manganate/periodate system or metal-catalyzed processes. Oxidative cleavage of olefins with tungstic acid or co-oxidation with oxone was successful with 80% yield of pelargonic acid and C13 acid. A mixture of glycerol (200 mg, 2.2 mmol), Aza (98%, 305 mg, 1.1 mmol), BrA (99%, 620 mmol, 2.2 mmol) and p-toluene sulfonic acid (20 mg) were heated at 140° C. for 5 h under a stream of nitrogen. The reaction was cooled and purified by flash chromatography (5% ethyl acetate/hexane) to yield the pure triglycerides as a colorless oil. BrA obtained by oxidation of eruric acid—(˜50%) obtained from rapeseed oil by oxidative cleavage of raw rapeseed oil with tungistic acid H2OWO4, resulted in high yield mix of PA and BrA at 80° C. during oxygen bubbling or oxygen 10 bar pressure for 12 hours.

### Example 24: Esterification Synthesis of TriAza: Quality Control

Screening of end products of reaction after process was done in ozonolysis/oxidation (acidic pH) vs oxidation (acidic pH) only and ozonolysis/oxidation (at neutral or basic pH) reactions exemplified in Table 12 below, proving that TriAza can be obtained at acidic oxidation only, otherwise hydrolysis of TG results in FA release even if desaturation of bonds resulted in azelaic acid formation, glycerol bond was fully hydrolyzed. Therefore—PA is essential by-product and can serve as identity specification in QC parameters, but Aza is an indicator of resulted reaction not properly processed and can serve as impurity specification for QC realease.

TABLE 12
Batch process conditions and end products

| Batch process/end | | | | |
|---|---|---|---|---|
| products | Oleic | azelaic | Pelargonic | TriAza |
| Ozonolysis/H2O2/Asc | − | − | + | + |
| Pelargonic/separation | − | − | + | + |
| Ozonolysis/O2- | − | − | + | + |
| propionic/succinic | | | | |
| acid, pH < 6-5,5 | | | | |
| Ozonolysis/oxidation, | + | + | + | − |
| pH 7-8 | | | | |

### Example 25: Testing Minimal Inhibitory Concentration to P. acnes by Microdilution

P. acnes after double passage on Chocolate plates (Novamed) were grown in anaerobic conditions at 37ºC for 1-2 days. The bacteria were Gram stained and the proper morphology of the bacteria was assured. Then, bacteria were resuspend in Thioglycolate medium (HyLabs) at concentration of 0.02 O.D.₆₀₀. Meanwhile, ELISA plates containing at the first raw 200 ul of undiluted tested material were prepared. The rest wells were filled with 100 ul of Thioglycolate medium and 11 serial ½ dilutions by transferring 100 ul of undiluted material was performed. Once the ELISA plate was ready, 100 ul of prepared bacterial culture was added. Bacteria alone and medium w/o bacteria were used as standard controls. The plates were incubated in anaerobic conditions at 37° C. for 2 days. Minimal inhibitory concentration was determined visually and spectrophotometrically, while minimal bactericidal concentration was determined after plating of the bacteria on solid medium (Chocolate plates). On the plates bacteria were incubated in anaerobic conditions at 37° C. for 2 days and the number of appeared colonies were counted manually.

### Results

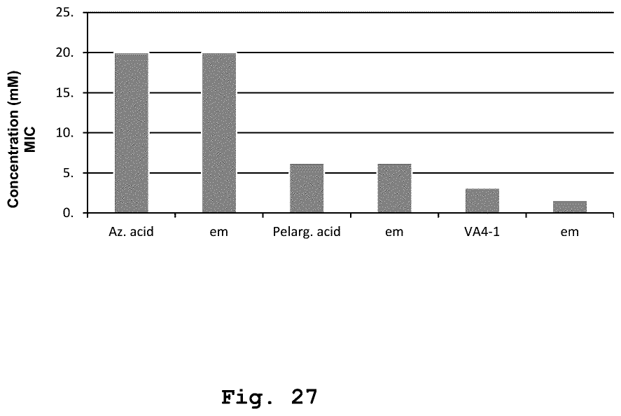

Screening antibacterial efficacy of C9-OCFA (azelaic-Az. Acid and Pelargonic-Pelarg. Acid) and MOCT-TriAza (batch VA4-1) with and without emulsifier, as shown in FIG. 27, proves that TriAza has the highest anti-growth bacteriostatic activity. MIC bacteriostatic activity of TriAza only, not C9 FA, is enhanced by use of surfactant or emulsifier, where 1 mM is sufficient to stop growth of the pathogen. Therefore, TriAza formula will essential comprise “green” surfactant emulsifier in the disclosed composition

TABLE 13
Anaplerotic C999 vegetable oil- based Formulas
for Biopotency and Efficacy Experiments

| Olebiome | | | |
|---|---|---|---|
| Composition | Formula 1 | Formula 2 | Formula 3 |
| Carrier oil | 70% Jojoba | 70% Olive | 70% Argan |
| comprising | oil/Sunflower | oil/almond | oil/sesame |
| triolein at | oil (50-50) | oil (60/30) | oil (80/20) |
| least 20% | | | |
| TriAZA/ | TriAza | Tg9 + Tg7 | TriAza/C9 |
| MOCFA/OCFA | 8%/Pelargonic | (50-50) 10%/isoC9 | diester- |
| | 10% | ester/diester | DiAza 10% |
| | | mix14% | |
| Ascorbyl | 10% | 5% | 10 |
| palmitate | | | |
| CoQ10 | 1% | 1% | — |
| Vitamin E | | | 10 |
| Acyl- | 1% | | |
| carnitine | | | |

### Example 26: Method of Testing the “Aged Appearance” Status of the Skin

Method of subjective qualitative evaluation of skin whitening and smoothening at the test site and improvement in fine lines is evaluated subjectively by the investigator and/or by the subjects in accordance with the following scoring pattern: −3=marked deterioration; −2=moderate, visibly uneven deterioration; −1=slight deterioration; 0=no perceptible change or improvement; 1=slight change or improvement; 2=moderate change or improvement (whitening; perceptible and visible change, with less than 50% lightening of skin color); 3=marked improvement or remarkable change or improvement (whitening; very visible change with even and uniform skin whitening covering more than 80% of the contact area). Three study volunteers used TriAza for reduction of skin “aged appearance”—all three after month of use reported 2-3 scale of appearance improvement.

### Example 27: Method of Measuring Quantitative Skin Quality Under Ongoing Evaluation

Methods of quantitative skin quality under ongoing evaluation including transepidermal water loss (TEWL), is measured on the cheeks with a Tewameter® TM300 (Courage+Khazaka ElectronicGmbH), which measures water evaporation from the skin. For normal skin, under ambient conditions, TEWL oscillates between 4 and 10 g/h/m². This water loss accounts for a total of about 500 ml per day but may increase up to 30 times higher when the epidermis is damaged. Therefore, TEWL correlates with skin barrier function and can be a measure of dysfunction. TEWL is regarded as an important parameter when measuring skin barrier integrity.

### Example 28: Method of Measuring Moisture Level or Hydration State of the Skin of the Face

The moisture level or hydration state of the skin of the face is measured with a Corneometer® (Courage+Khazaka Electronic GmbH, Cologne, Germany). As with the Mexameter®, the test site is the cheekbone area. The Corneometer® uses capacitance to measure the moisture content of the stratum corneum.

### Example 29: Method of Measuring Aging Appearance Using the Grade of Crow's Feet Wrinkles

Measurement of aging appearance using the grade of Crow's feet wrinkles (GCFW) is a standardized method by 1-6 point scale skin smoothening. GCFW, i.e. “crow's feet” at the outer corner of the eye can be determined by clinical scoring of the Crow's feet wrinkles. Alternatively, digital imaging systems to test site for wrinkle reduction and SkinSys software (Sometech Inc, Seoul, South Korea) and a Coccam digital camera (Beauty Korea World Co Ltd, Seoul, South Korea) are used. SkinSys is professional skin analysis and treatment software that enables the user to perform a well-organized scientific evaluation of cosmetic products. The skin analysis and measuring functions include: 1) 3D analysis of skin curvature (wrinkle reduction); and 2) analysis of keratin content by using a precise edge-detection algorithm. To test effect of wrinkles subdermal injection of TriAza Formulas 1-3 (Table 13) are coadminstrated with botox and hyaluronic acid and elasin for evauation.

### Example 30: Method of Measuring Skin Elasticity

Skin elasticity is quantified with a Triplesense TR-3 sensor scan device (Schott Moritex Corporation, Saitama, Japan). To test elasticity, samples of Olebiome C999 formula OCFA/MOCT Formula 2 was preconditoning for topical administration of low molecular weight hyaluronic acid for evaluation.

### Example 31: Method of Measuring Colorimetric Skin-Whitening Efficacy

Colorimetric skin-whitening efficacy are quantified by means of the melanin index using a Mexameter® MX18 (Courage+Khazaka Electronic GmbH, Cologne, Germany). Whitening efficacy is tested in the cheekbone area. The melanin index recorded is the average of three readings. The measurement is based on the absorption principle. The probe emits light of three defined wavelengths and a receiver measures the light reflected by the skin and thus the light absorbed. The melanin is measured by using two of the three wavelengths, which are chosen to correspond to the different rates of light absorption by the melanin pigments. Formula 1 (Table 13) of Olebiome C999 with Asc acid, CoQ10 and Acyl carnitine are provided below.

### Example 32: Wound Healing Compositions

The wound healing compositions of Table 13 is formulated for cell culture experiments and may be utilized in topical products, ingestible products, and tissue culture medium to protect mammalian cells and increase the resuscitation rate of injured mammalian cells.

### Example 33. Cell Culture Experiment of Olebiome TriAza-Measuring Reduced Levels of Hydrogen Peroxide

Mammalian epidermal keratinocytes can be employed to examine the ability of various antioxidants to reduce levels of hydrogen peroxide in these cells. Hydrogen peroxide levels are measured after the cells are exposed to ultraviolet light in the wavelength range from 290 to 320 nm (UV-B) or to the inflammatory compound 12-0-tetradecanoyl-phorbol-13-acetate (TPA). TriAza Formula 1 and 3 at concentration 0,5-1% of culture media dish and can be tested at various concentrations to determine the effect of concentrations of this antioxidant on the hydrogen peroxide production by epidermal cells. Mammalian epidermal keratinocytes are isolated by trypsinization of epithelial sheets and grown in modified basal MCDB 153 medium supplemented with epidermal growth factor, 10% fetal calf serum and hydrocortisone. Cells are maintained in a humidified incubator with 5% carbon dioxide at 37° C. Keratinocytes are seeded in 60 mm culture dishes at a cell density of 3×10 cells per dish and the cultures are exposed to 1 M.E.D. dose of ultraviolet-B light (100 mj/cm) or treated with 100 ng/ml of TPA. TriAza cell culture. Formulas 1-3 (see Table 13) are dissolved in 2% DMSO and 2% surfactant (PVP). The appropriate concentration of test solution or combination of test solutions are added to the cells immediately prior to exposure of the cells to ultraviolet light-B or TPA [100 ng/ml]. Stock solutions are prepared so that the vehicle did not constitute more than 1% of the total volume of the culture media.

Intracellular hydrogen peroxide production by mammalian epidermal keratinocytes cab be measured using dichlorofluorescein diacetate (DCFH-DA, Sigma). DCFH-DA is a non-polar non-fluorescent compound that readily diffuses into cells where it is hydrolyzed to the polar non-fluorescent derivative DCFH which then becomes trapped within the cells.

In the presence of intracellular hydrogen peroxide, DCFH is oxidized to the highly fluorescent compound DCF. Hence, cellular fluorescence intensity is directly proportional to the level of intracellular hydrogen peroxide produced. Cellular fluorescence intensity can be monitored by fluorimetry and by flow cytometry. Mammalian epidermal keratinocytes (1×10⁶ per dish) are incubated at 37° C. with 5 uM of DCFH-DA. Production of hydrogen peroxide is measured using a Coulter Profile analytical flow cytometer. Each analysis is repeated three times and the quantitation of fluorescence is expressed in terms of femtomoles (fmol, 10 moles) of DCF oxidized per cell, which is a direct measure of the intracellular hydrogen peroxide produced. All comparisons are assessed against the controls, which produced 250 hydrogen peroxide-H₂O₂ fmol/cell. The positive numbers represent H₂O₂ production in excess of the control and the negative numbers represent H₂O₂ production below the control.

C999 composition of Formula 1-Table 13 resulted in 2,5-5 folds reduction of H₂O₂ production in comparison to control, for 0,5-1% concentration of the TriAza comprising formulas (Formula 3-Table 13) only at concentration 0,25%-0,5% resulted in same range of activity as C999 Olebiome (Formula 2-Table 13).

### Example 34. Alternative Cell Culture Experiment of Olebiome TriAza-Measuring Reduced Levels of Hydrogen Peroxide

Additional experiments of protective epidermal cell barrier effect can be performed by measurement of TBAR activity under divalent iron induced oxidative stress resulted in lipid peroxidation (LPO) ex vivo. Human keratinocytes tissue (Mattek, EpiOral) culture are preincubated with Formula 1, Formula 2 and Formula 3 (Table 13) during 30 min at 37° C. Afterwards, freshly prepared FeSO₄·7H₂O are added to induce lipid peroxidation and slices are incubated for another 15 min at 37° C. with gentle shaking under permanent oxygen aeration. At the end of the incubation period, medium is separated from the tissue by centrifugation at 3500×g for 5 min and 0.5 ml of medium is taken for thiobarbituric acid-TBA reactive substance (thiobarbituric acid reactive substance-TBARS) content determination. Not treated control under LPO stress is evaluated as 100% of oxidative stress damage. The most prominent inhibition of TBARS production (about 60%) is observed after Formula 3 (reasonable impact of vitamin E), Formula 1 and 2 results in 25% and 40% TBARS reduction respectively.

### Example 35. Examination of Olebiome™® Compositions TriAza-Formula 1 and C999 MOCT/OCFA-Formula 2 on Functionality Assay for Wound Gap Closure-Scratch Assay

In vitro wound healing assay can be performed by measuring percent gap open for a wound created on fully confluent culture of primary gingival keratinocytes (ATCC® PCS-200-014™) treated with Formula 1 and 2. 1% of total culture media dish is added from each Formula 1, 2 and 3 dissolved in DMSO and prepared as described above. Images are acquired at 8; 12; 16 and 24 hrs, post administration under a phase contrast microscope. Media only on gap closure is utilized as control. For the control group the wound gap closed at <50% at 24 hrs. For Formula 1 and 3 treatments >80% closure is seen in 12 hours, 100% closure is seen at 16 hrs. For Formula 2 and 3>80% closure is seen at 24 hrs. All Formulas have wound closure potential for the protection of epidermal mammalian cells barrier.

### DISCUSSION

The invention, in utilizing topical compositions for the treatment of lipase virulent factor overexpressed pathogenic microbiome such compositions preferably utilizing as active ingredient azelate esters, such as tri-azelate glycerol, alone, or in combination with other active ingredients. TG mimetic is composed from Azelaic acid instead of oleic fatty acid. It was surprisingly found that by oxidation of Triolein containing substrate-triazelaine as a prodrug precursor of AzA was obtained, and can be released by triglyceride lipases in GI system or by microbiome releasing TG lipase into host extracellularly, thus inducing Azelaic acid release form TriAzA. The antimicrobial activity of TriAzA was unexpectedly higher in comparison to equimolar AzA, and surprisingly, had no effect on non-virulent microbiome. The disclosed process of oxidative cleavage of double bonds of Triolein or olive oil containing Triolein, resulted in industrially friendly methods of production of glycerol of azelaic acids, named TriAza, or triazelaine, that can serve as a metabolic substrate for microbial lipase. This way, a control over lipase, a major virulent factor overexpressed in pathogenic microorganisms, can be achieved. The observed selectivity and specificity to virulent lipase producing microbiome, allows to maintain control of pathogenic and opportunistic infections and pathologies without causing damage to host microbiome. The disclosed invention thus has a superior properties in terms of biocontrol (pathogens vs host microbiome). In addition, the disclosed compositions may deliver donor or cultured microbiome and confer superior preservation. In addition, a superior MIC as compared to the Azelaic acid and MBC potency were observed in several lipase producing species, including resistant planktonic pathogens. Microbiome regulating effect of Triaza provides multifunctional benefit to health span enhancement for various indications in human organism.

In addition, the disclosed invention provides superior topical anaplerotic compositions based on TriAza and/or C9 MOCT/OCFA compositions. Surprisingly, the compositions of the invention exhibit a plurality of functions: (1) regulation of cellular barrier and anti-inflammatory activity; (2) preserving cellular energetic status based on anaplerotic nature of odd chain fatty acids as substrate to Krebs cycle regeneration and as a medium chain fatty acid source for ketogenic energy; (3) microbiome balance through preventing overgrowth of lipase overexpressing commensal microbiome (Staph spp., Candida) and pathogenic species (Aspergillus; MRSA; Pseudomonas). Therefore, such multifunctionality targets three major skin ageing causes: (1) skin barrier loss, translated in aging appearance related symptoms—skin dryness, pigmentation, wrinkles, and loss of elasticity; (2) mitochondrial dysfunction and, (3) microbiome disbalance that also impact “aged appearance” of mature skin.

According to some embodiments, the invention provides a method of delivering at least one chemical entity and/or biomolecule into a food product for consumption or a raw material for the preparation of said food product, comprising adding to said food product or the raw material for the preparation of said food product an effective amount of the composition according to the embodiments of the invention. In one embodiment, said food product is a product for human consumption. In one embodiment, said food product is an animal feed.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will prevail. In addition, the materials, methods, and examples are illustrative only and are not intended to be necessarily limiting.

The terminology used herein is for the purpose of describing particular embodiments only and is not intended to be limiting of the invention. As used herein, the singular forms “a,” “an” and “the” are intended to include plural forms as well, unless the context clearly indicates otherwise.

It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their “including but not limited to”. The term conjugates mean “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

As will be appreciated by those of skill in the art, the compounds of the various formulas disclosed herein may contain chiral centers, e.g., asymmetric carbon atoms. Thus, the present disclosure is concerned with the synthesis of both: (i) racemic mixtures of the active compounds, and (ii) enantiomeric forms of the active compounds. The resolution of racemates into enantiomeric forms and racemization of optically active enantiomeric form can be done in accordance with known procedures in the art. Geometric isomers of double bonds and the like may also be present in the compounds disclosed herein, and all such stable isomers are included within the present disclosure unless otherwise specified. Also included in the compounds of the disclosure are tautomers (e.g., tautomers of triazole and/or imidazole) and rotamers. All chains defined by the formulas herein which include three or more carbons may be saturated or unsaturated unless otherwise indicated.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

An “optionally substituted” group refers to a functional group in which one or more bonds to a hydrogen atom contained therein are replaced by a bond to non-hydrogen or non-carbon atoms, provided that normal valences are maintained and that the substitution results in a stable compound. Substituted groups also include groups in which one or more bonds to a carbon (s) or hydrogen (s) atom are replaced by one or more bonds, including double or triple bonds, to a heteroatom. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different. In choosing the compounds of the present invention, one of ordinary skill in the art will recognize that the various substituents are to be chosen in conformity with well-known principles of chemical structure connectivity.

As used herein, “H” refers to a hydrogen atom. “C” refers to a carbon atom. “N” refers to a nitrogen atom. “O” refers to an oxygen atom. “Halo” refers to F, Cl, Br or I. The term “hydroxy,” as used herein, refers to an —OH moiety. “Br” refers to a bromine atom. “Cl” refers to a chlorine atom. “I” refers to an iodine atom. “F” refers to a fluorine atom. An “acyl group” is intended to mean a group —C(O)—R, where R is a suitable substituent, for example, an acetyl group, a propionyl group, a butyroyl group, a benzoyl group, or an alkylbenzoyl group. “Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). In some embodiments the alkyl can be a lower alkyl. “Lower alkyl” refers to straight or branched chain alkyl having from 1 to 3, or from 1 to 5, or from 1 to 8 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. As used herein, the identification of a carbon number range, e.g., C1-C12 alkyl, is intended to include each of the component carbon number moieties within such range, so that each intervening carbon number and any other stated or intervening carbon number value in that stated range is encompassed, such that sub-ranges of carbon number within specified carbon number ranges may independently be specified. For example, C1-C12 alkyl is intended to include methyl, ethyl, propyl, butyl, pentyl, hexyl, heptyl, octyl, nonyl, decyl, undecyl and dodecyl, including straight chain as well as branched groups, as noted above, and the carbon number range C1-C12 alkyl may also be more restrictively specified as sub-ranges such as C1-C4 alkyl, C2-C8 alkyl, C2-C4 alkyl, C3-C5 alkyl, or any other sub-range within the broader carbon number range. In addition, ranges of carbon numbers specifically excluding a carbon number or numbers are contemplated, as are sub-ranges excluding either or both of carbon number limits of specified ranges. As generally understood by those of ordinary skill in the art, “saturation” refers to the state in which all available valence bonds of an atom (e.g., carbon) are attached to other atoms. Similarly, “unsaturation” refers to the state in which not all the available valence bonds are attached to other atoms; in such compounds the extra bonds usually take the form of double or triple bonds (usually with carbon). For example, a carbon chain is “saturated” when there are no double or triple bonds present along the chain or directly connected to the chain (e.g., a carbonyl), and is “unsaturated” when at least one double or triple bond is present along the chain or directly connected to the chain (e.g., a carbonyl). Further, the presence or absence of a substituent depending upon chain saturation will be understood by those of ordinary skill in the art to depend upon the valence requirement of the atom or atoms to which the substituent binds (e.g., carbon). “Alkenyl,” as used herein, refers to a straight or branched chain hydrocarbon containing from 1 or 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Alkynyl,” as used herein, refers to a straight or branched chain hydrocarbon group containing from 1 or 2 to 10 or 20 or more carbon atoms, and containing at least one carbon-carbon triple bond. Representative examples of alkynyl include, but are not limited, to acetylenyl, 1-propynyl, 2-propynyl, 3-butynyl, 2-pentynyl, 1-butynyl and the like. The term “cycloalkyl,” as used herein, refers to a saturated cyclic hydrocarbon group containing from 3 to 8 carbons or more.

It will be understood that the compounds, compositions and methods provided herein may be further specified in some embodiments by provisos or limitations excluding specific substituents, groups, moieties, structures, ingredients, steps, or conditions, as applicable, in relation to various broader specifications and exemplifications set forth herein.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein the term “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

“Treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms, or relieving the disease, i.e., causing regression of the disease or its clinical symptoms.

A “therapeutically-effective amount” or an “effective amount” means the amount of a compound or a dosage form that, when administered to a subject for treating a disease, is sufficient to effect such treatment for the disease. The “therapeutically-effective amount” will vary depending on the compound, the disease, and its severity and the age, weight, etc., of the subject to be treated.

A “individually therapeutically effective amount” means an amount of a single active agent that, when administered, is effective to treat inflammation. The efficacy of an individually effective amount of an active agent can be supplemented with, or synergistically enhanced by, the addition of the second active agent. The second active agent may be administered in an individually therapeutically effective amount or as part of a combined composition of therapeutically effective amount.

As used herein the term “Pharmaceutically acceptable salt” refers to salts, which retain the biological effectiveness and properties of compounds which are not biologically or otherwise undesirable. Pharmaceutically acceptable salts refer to pharmaceutically acceptable salts of the compounds, which salts are derived from a variety of organic and inorganic counter ions well known in the art.

The pharmaceutical dosage forms may be prepared as medicaments to be administered orally. Suitable forms for oral administration include, without limitation, solutions, syrups and suspensions; such as ready-to-use syrups and suspensions, or reconstituted from solid dosage form such as, without limitation, dry powder. The dosage form may contain suitable binders, lubricants, coloring agents, flavoring agents, flow-inducing agents, stabilizing agents, solubilizing agents, antioxidants, buffering agent, chelating agents, and fillers, all collectively or individually fall under the definition of the term “pharmaceutically acceptable carrier” or “pharmaceutically acceptable excipient”. For oral administration in the dosage form, the active drug component can be combined with an oral, non-toxic, pharmaceutically acceptable, inert filler such as gelatin, agar, starch, methyl cellulose, mannitol, sorbitol, and the like. Suitable binders include starch, gelatin, natural sugars such as corn starch, natural and synthetic gums such as acacia, tragacanth, or sodium alginate, povidone, cellulose based soluble polymers such as but not limited to hydroxypropylomethylcellulose, polyethylene glycol, and the like. Glidants used in these dosage forms include sodium benzoate, sodium acetate, polyethylene glycole, like. Stabilizing and the (antimicrobial) agents include benzoic acid, and salts thereof, parahydroxybenzoate and salts thereof, sorbic acid and salts thereof and the like. Stabilizing (physical) agents include viscosity enhancing polymers such as hydroxyethyl cellulose, xanthan gum and the like.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

1. A composition comprising triazelaine and, optionally, a carrier.

2. The composition of claim 1, further comprising one or more active agent.

3. The composition of claim 2, wherein the one or more active agent is selected from the group consisting of a biomolecule, a sample of microbiome, an anti-microbial agent, an anti-aging agent, an anti-viral agent, an anti-fungal agent, an anti-bacterial agent, an antioxidant, an anti-inflammatory agent, an antibiotics, an antiparasitic agent, an anesthetic agent, an analgetic agent, an antiallergic agent, an antipruritic agent, an immunosuppressant, an anti-angiogenic agent, a vasoconstrictor, a probiotic agent, a sample of non-lipase probiotic microorganisms, or a mixture thereof.

4. The composition of claim 3, wherein the biomolecule is selected from the group consisting of deoxyribonucleic acid (DNA), ribonucleic acid (RNA), organic molecule, inorganic molecule, amino acid, vitamin, polyphenol, steroid, peptide, polypeptide, and protein complexes.

5. The composition of claim 3, wherein the sample of microbiome is donor-autologous microbiome, allogenic microbiome, or in-vitro cultured microbiome.

6. The composition of claim 1, suitable for a topical administration.

7. The composition of claim 1, wherein the composition is a solid composition, a semi-solid composition, or a liquid composition.

8. The composition of claim 1, selected from the group consisting of powder, suspension, emulsion, cream, paste, gel, suppository, ointment, spray, foam, soap, shampoo, mill, colloid, and oil.

9. The composition of claim 1, further comprising at least one of a surfactant, a preservative, and a colorant.

10. The composition of claim 1, characterized by triazelaine content in the range of 1% to 100%.

11. A method of treating an inflammatory condition of the skin in a subject in need of such treatment, comprising administering to the subject an amount of triazelaine effective to treat said condition.

12. The method of claim 11, inflammatory condition of the skin is selected from the group consisting of acne, eczema, acne, atopic dermatitis, tissue necrosis, skin sores, psoriasis, cellulitis, fungal infection, gangrene, and disorder of the pilosebaceous unit.

13. A method of slowing progression of an inflammatory condition of the skin in a subject, comprising administering to the subject an effective amount of the composition of claim 1.

14. The method of claim 13, wherein the inflammatory condition of the skin is selected from the group consisting of acne, eczema, acne, atopic dermatitis, tissue necrosis, skin sores, psoriasis, cellulitis, fungal infection, gangrene, and disorder of the pilosebaceous unit.

15. A method of treating an inflammatory condition of the skin in a subject, comprising administering to the subject an effective amount of the composition of claim 1.

16. The method of claim 15, inflammatory condition of the skin is selected from the group consisting of acne, eczema, acne, atopic dermatitis, tissue necrosis, skin sores, psoriasis, cellulitis, fungal infection, gangrene, and disorder of the pilosebaceous unit.

17. A method of treating an inflammatory condition of the skin associated with a lipase-producing microorganism, comprising administering to the subject an amount of triazelaine effective to treat said condition.

18. The method of claim 17, wherein the lipase-producing microorganism is selected from the group consisting of Pseudomonas spp, Cutibacterium acnes, Staphylococcus, Candida, Malazzeroa, and Corynebacterium.

19. A method of balancing human microbiome in a subject in need, comprising administering to the subject in need an effective amount of the composition of claim 1.

20. A method of preventing aging of the skin in a subject, comprising administering to the subject the composition of claim 1.
